(19) United States
(12) Patent Application Publication
(10) Pub. No.: US 2026/0060924 A1
(43) Pub. Date: Mar. 5, 2026

(54) SEMI-SOLID ORAL DOSAGE FORM WITH ENCAPSULATED ACTIVE INGREDIENT

(71) Applicant: Concept Matrix Solutions, Newbury Park, CA (US)

(72) Inventors: Robert Davidson, Woodland Hils, CA (US); David Reid, Woodland Hills, CA (US); Tony LaRosa, Woodland Hills, CA (US); Sevan Kesenci, Woodland Hills, CA (US)

(21) Appl. No.: 19/315,496

(22) Filed: Aug. 30, 2025

Related U.S. Application Data
(60) Provisional application No. 63/689,152, filed on Aug. 30, 2024.

(51) Int. Cl. A61K 9/00; A61K 9/48; A61K 45/06

(52) U.S. Cl. CPC A61K 9/006; A61K 9/0056; A61K 9/4816; A61K 9/4833; A61K 45/06

(57) ABSTRACT

A semi-solid oral dosage form that includes one or more excipients, an active ingredient, and a polymer. The polymer encapsulates at least a portion of the active ingredient, and the portion of the active ingredient that is encapsulated does not come in direct contact with the one or more excipients present in the semi-solid oral dosage form. The semi-solid oral dosage form can be, e.g., an oral thin film (OTF) or gummy. Also provided is a method of orally administering the semi-solid oral dosage form, as well as methods of manufacturing the semi-solid oral dosage form.

### CROSS-REFERENCE TO RELATED APPLICATIONS

This application claims the benefit of priority to U.S. Provisional Patent Application No. 63/689,152, filed Aug. 30, 2024, the contents of which are incorporated herein by reference in their entirety.

### BACKGROUND

The delivery of active pharmaceutical ingredients (APIs) via semi-solid oral dosage forms, such as gummies and oral thin films (OTFs), has gained increasing popularity due to patient preferences for palatable, easily administered alternatives to traditional tablets and capsules. These formats offer unique benefits including improved patient compliance, the potential for rapid onset of action, and the ability to accommodate individuals with swallowing difficulties or aversions to conventional solid dosage forms. However, the adoption of gummies and OTFs for a broader range of APIs has been severely constrained by challenges associated with API stability, particularly in the presence of water, acidic excipients, oxygen, light, and other destabilizing agents encountered during manufacturing, storage, and administration.

APIs such as peptides, enzymes, certain vitamins, probiotics, and a variety of small molecules are known to be highly sensitive to moisture, acidic environments, oxidative conditions, or UV exposure, leading to rapid degradation, reduced potency, and diminished shelf-life. Furthermore, APIs with unpleasant tastes or odors pose significant obstacles to patient acceptance when formulated in dosage forms intended for oral dissolution or mastication, as is typical for gummies and OTFs. Conventional approaches to taste-masking and stabilizing sensitive APIs—such as enteric coatings, specialized packaging, or chemical modification—are often unsuitable, impractical, or cost-prohibitive for soft, flexible, or fast-dissolving dosage matrices. These limitations have significantly narrowed the scope of APIs that can be effectively delivered using semi-solid oral systems, preventing full realization of patient-centric advantages offered by these formats.

### SUMMARY

The present invention demonstrates surprising and unexpected results in stabilizing a wide range of active pharmaceutical ingredients (APIs) that would otherwise be highly vulnerable to degradation during manufacturing, storage, and use in semi-solid oral dosage forms such as gummies and oral thin films (OTFs). It was previously believed that incorporating water-, acid-, oxygen-, UV-, or excipient-sensitive APIs into moisture-containing, soft, or flexible dosage forms was impractical or impossible without extensive degradation, poor shelf-life, or loss of potency. The use of a tailored encapsulation step prior to API incorporation into the gummy or OTF matrix, as described herein, effectively isolates the APIs from destabilizing excipients, water, and acidic microenvironments present in the matrix. As a result, APIs previously prone to rapid degradation—including highly labile small molecules, peptides, probiotics, or vitamins—are rendered stable throughout conventional gummy or OTF manufacturing processes and under typical storage conditions, which was unexpected given the hydrophilic and sometimes chemically reactive environment of these drug delivery platforms.

Notably, the encapsulation strategy provided for the protection of APIs was found to confer far more robust protection than predicted based on analogous prior art systems. For example, APIs susceptible to hydrolysis, oxidation, or acid-induced decomposition not only maintained their structural integrity during the high-humidity and thermal conditions inherent to gummy or film manufacturing, but also retained potency over extended storage, even when exposed to environmental stresses such as temperature fluctuations and light. This outcome was particularly surprising for APIs sensitive to both water and acid, as conventional wisdom held that even minute exposure to these destabilizers-such as from residual process water or co-formulated acids-would rapidly render many APIs inactive. Contrary to such expectations, the protective polymer coat acts as a highly effective barrier, preserving both chemical and physical stability and allowing for the practical, scalable production of potent, orally acceptable gummies and OTFs containing these sensitive APIs.

Beyond stability, the invention also unexpectedly resolves longstanding issues with taste masking and palatability for APIs with inherently bitter, metallic, or otherwise objectionable flavors. By encapsulating the API, not only is its chemical stability maintained, but its taste and odor are physically isolated from the oral cavity and sweetener matrix, dramatically improving patient acceptability without the need for excessive flavoring additives or sophisticated masking agents. This synergistic stabilization and taste masking effect, achieved simply through pre-encapsulation in a broad array of matrices and processing conditions, was unforeseen and enables the expansion of effective, patient-friendly dosage forms into therapeutic areas previously considered impractical or unattainable for semi-solid oral formats.

There remains a significant unmet need for compositions and methods that enable the stable incorporation of a wide variety of APIs, including those that are water-, acid-, oxygen-, and light-sensitive, into semi-solid oral dosage forms such as gummies and OTFs, while also effectively addressing taste and odor challenges. The present invention satisfies these needs and overcomes the prior art limitations through the development of an encapsulation approach that protects the API from its environment, thereby preserving stability, potency, and acceptability in dosage forms previously considered unsuitable for such APIs.

The present invention provides a semi-solid oral dosage form that includes one or more excipients, an active ingredient, and a polymer. The polymer encapsulates at least a portion of the active ingredient, and the portion of the active ingredient that is encapsulated does not come in direct contact with the one or more excipients present in the semi-solid oral dosage form. The semi-solid oral dosage form can be, e.g., an oral thin film (OTF) or gummy.

The present invention provides a semi-solid oral dosage form comprising one or more excipients, an active ingredient, and a polymer, wherein the oral dosage form is selected from an oral thin film (OTF) and a gummy and the polymer encapsulates at least a portion of the active ingredient, the portion of the active ingredient that is encapsulated does not come in direct contact with the one or more excipients.

### DETAILED DESCRIPTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “weight percent” (sometimes written as wt. % or % w/w) expresses the concentration of a component in a mixture as the number of grams of that component per 100 grams of the total mixture.

The term “oral thin film (OTF)” refers to a flexible, film-shaped, semi-solid oral dosage form that comprises a polymeric matrix and is designed to dissolve, disintegrate, or release its contents when placed in the oral cavity or upon swallowing, optionally adhering to a mucosal surface. The film may contain one or more active pharmaceutical ingredients, excipients, and additional functional agents, and may be formulated for immediate, modified, or controlled release.

The term “gummy” refers to a semi-solid or gelatinous oral dosage form, typically chewy or elastic in texture, formed from a matrix of one or more gelling agents (such as gelatin, pectin, agar, carrageenan, or synthetic polymers), and containing one or more active pharmaceutical ingredients and excipients. Gummies are typically ingested orally and may be formulated for various release profiles or taste masking.

The term “pH” refers to the negative logarithm of the hydrogen ion activity in a solution, representing the degree of acidity or basicity, on a standard scale ranging from 0 to 14, with 7 being neutral, lower values being more acidic, and higher values more alkaline.

The term “pH-sensitive polymer” refers to any natural, synthetic, or chemically modified polymer whose solubility, swelling, permeability, or structural integrity is significantly altered by the pH of its environment. Such polymers are engineered to remain intact or resist dissolution at certain pH conditions (e.g., the acidic stomach), and become soluble, swell, degrade, or otherwise alter to release an active ingredient when the pH crosses a target threshold (e.g., in the intestine or colon), thereby enabling site-specific, pH-triggered, or delayed release of pharmaceuticals or nutraceuticals in the gastrointestinal tract.

The term “intestine” refers to the portion of the gastrointestinal tract extending from the pyloric sphincter of the stomach to the anus, encompassing the small intestine (duodenum, jejunum, and ileum) and the large intestine (colon, rectum), and serving as the site for nutrient absorption and further digestion.

The term “colon” refers to the large intestine distal to the ileocecal valve, including the ascending, transverse, descending, and sigmoid segments, and extending up to the rectum, primarily responsible for reabsorption of water and electrolytes, and for fecal storage.

The term “cellulose derivative” refers to any compound based on the natural polysaccharide cellulose that has been chemically modified, including but not limited to hydroxypropyl methylcellulose, ethylcellulose, methylcellulose, cellulose acetate phthalate, and carboxymethyl cellulose. Such derivatives may possess altered solubility, film-forming, or gelation properties useful for pharmaceutical formulation.

The term “methacrylic acid copolymers” refers to synthetic polymers formed by copolymerization of methacrylic acid and one or more comonomers (such as methyl methacrylate or ethyl acrylate), which may include, without limitation, commercially available grades known as Eudragit. Such copolymers are used in oral pharmaceutical dosage forms for enteric, pH-responsive, or sustained release applications.

The term “synthetic polymer” refers to polymers that are manufactured through chemical synthetic processes, as opposed to being naturally derived without modification. Synthetic polymers include, but are not limited to, methacrylic acid copolymers, polyvinyl alcohol, polyethylene glycol, polyurethanes, polyvinylpyrrolidone, and others used in pharmaceutical formulations.

The term “time dependent polymer” refers to polymers whose physicochemical behavior, including swelling, dissolution, or erosion, is modulated over a predictable time frame upon exposure to a biological fluid, thus enabling temporal control of release or protection of ingredients independent of local pH or enzymatic activity.

The term “degrade” refers to any process by which a substance, such as a polymer or active pharmaceutical ingredient, undergoes chemical or physical breakdown into its constituent components or into other, smaller or less complex molecules, as a result of exposure to conditions including, but not limited to, water, acids, bases, heat, light, enzymes, or oxidative agents.

The term “enzymes” refers to naturally occurring or synthetic proteins or catalytic molecules that accelerate specific biochemical reactions, often involved in the degradation, transformation, or activation of pharmaceutical ingredients or excipients within a biological system.

The term “lower gastrointestinal tract” refers to the portion of the gastrointestinal tract distal to the ligament of Treitz or duodenojejunal flexure, including the distal small intestine (ileum) and the entire large intestine (colon and rectum).

The term “encapsulated” refers to a physical form in which an active pharmaceutical ingredient or other material is at least partially enclosed or surrounded by a coating of a polymer or combination of materials, such that the core is physically separated from its immediate external environment and, optionally, protected from chemical, enzymatic, or physical destabilization until release is triggered.

The term “active pharmaceutical ingredient” or “API” refers to any compound, substance, or molecule that is intended to provide a desired therapeutic, preventive, or diagnostic effect when administered to a subject, whether alone or in combination with one or more excipients or other active substances.

The term “acidifying agent” refers to any compound or mixture added to a pharmaceutical composition for the purpose of lowering the pH, providing an acidic environment, or otherwise facilitating proton donation; examples include citric acid, tartaric acid, fumaric acid, malic acid, ascorbic acid, and hydrochloric acid.

The term “oral bioavailability (BA)” refers to the fraction or percentage of an administered dose of an active pharmaceutical ingredient that reaches the systemic circulation intact following oral administration, as measured by the area under the plasma concentration-time curve.

The term “UV light” refers to electromagnetic radiation having a wavelength in the range of approximately 100 to 400 nanometers, including ultraviolet A (320-400 nm), ultraviolet B (280-320 nm), and ultraviolet C (100-280 nm), exposure to which may cause degradation or transformation of susceptible pharmaceutical ingredients or polymers.

The term “oral dosage form” refers to any pharmaceutical composition designed for administration via the oral route, including but not limited to semi-solid formats such as gummies, oral thin films (OTFs), pastilles, chewable tablets, gels, or similar ingestible platforms.

The term “semi-solid” refers to a material having intermediate physical characteristics between a liquid and a solid, displaying shape retention yet possessing deformability or elasticity, typically with moisture content above standard solid dosage forms.

The term “excipient” refers to any pharmacologically inactive substance included in a dosage form to facilitate processing, stability, taste-masking, texture, or delivery of the active pharmaceutical ingredient, including but not limited to fillers, binders, solvents, flavoring agents, sweeteners, plasticizers, surfactants, colorants, or preservatives.

The term “potency” refers to the quantitative measure of biological or pharmacological activity of the active pharmaceutical ingredient in the dosage form, as determined using industry standard analytical or bioassay methods.

The term “ambient storage conditions” refers to the typical temperature and humidity environments encountered in commercial, distribution, retail, or home settings, generally within a range of about 15° C. to 30° C. and 30% to 65% relative humidity, though not limited thereto unless otherwise specified.

The term “direct contact” refers to a physical relationship in which two substances, such as an active pharmaceutical ingredient and an excipient, are not separated by a continuous barrier and thus are able to interact, exchange, or mix without obstruction, enabling chemical or physicochemical interaction or diffusion between the components.

The term “release type” (e.g., delayed, controlled, sustained, modified, pulsatile, targeted, extended, timed, site-specific release) refers to the engineered release of an active pharmaceutical ingredient according to a predetermined schedule, rate, or trigger that modifies the natural dissolution and absorption kinetics otherwise expected for the API in an immediate release format.

The term “biodegradable polymer” refers to any polymeric material capable of undergoing chemical, enzymatic, or biological breakdown into smaller units, monomers, or benign byproducts within a biological, physiological, or natural environment, and includes both synthetic and naturally derived polymers that degrade by hydrolysis, enzymatic cleavage, oxidation, or other mechanisms.

The term “water-sensitive API” refers to an active pharmaceutical ingredient that undergoes degradation, loss of potency, or chemical/physical transformation when exposed to water under typical processing or storage conditions.

The term “acid-sensitive API” refers to an active pharmaceutical ingredient that is susceptible to degradation, inactivation, or reduced efficacy upon exposure to acidic conditions, including those found in oral dosage matrices or gastric environments.

The term “oxygen-sensitive API” refers to an active pharmaceutical ingredient that undergoes oxidation or degradation upon exposure to ambient oxygen. The term “UV-sensitive API” refers to an active pharmaceutical ingredient that undergoes degradation, loss of activity, or structural change upon exposure to ultraviolet light at typical environmental intensities.

The term “functional group prone to hydrolysis” refers to any chemical moiety that can undergo cleavage or decomposition by reaction with water, including esters, amides, lactones, carbamates, anhydrides, imines, epoxides, or other relevant functional groups as applicable within the context of pharmaceutical chemistry.

The term “pharmaceutically acceptable” refers to substances, compositions, or conditions that meet safety, purity, efficacy, and regulatory standards for use in human or animal pharmaceutical applications as generally recognized by the FDA or equivalent regulatory authority.

The term “taste-masking” or “taste masking layer” refers to any physical, chemical, or structural means—such as encapsulation, coating, inclusion of barrier layers, or the integration of taste-masking excipients or additives—used in an oral dosage form to diminish, mask, or eliminate the perception of unpleasant or undesirable flavor, odor, or aftertaste associated with the active ingredient during oral administration.

The term “matrix” refers to the continuous phase of a semi-solid oral dosage form, comprising excipients, polymeric substances, and/or other functional agents in which active pharmaceutical ingredients—encapsulated or unencapsulated—are dispersed or embedded, providing mechanical cohesion, physical support, and delivery function to the dosage unit.

The term “barrier” or “protective barrier” refers to one or more layer(s), coating(s), or structural feature(s)—typically composed of polymeric or composite materials—applied to or surrounding an active pharmaceutical ingredient, excipient, or dosage form, that substantially limits or prevents the passage of destabilizing agents such as water, acids, oxygen, light, or co-formulated excipients, thereby maintaining the stability, potency, or integrity of protected materials under typical processing, storage, and use conditions.

The terms “permeability testing” and “tracer diffusion studies” refer to laboratory assessment methods that measure the degree or absence of molecular transfer (such as water, excipients, acids, or other small molecules) across a coating, encapsulation, or barrier, typically using tracers, dyes, labeled molecules, or analytical probes under controlled conditions that simulate storage, processing, or administration environments.

The term “encapsulation integrity” refers to the structural wholeness, continuity, and functional preservation of the encapsulating layer(s) or barrier around an active pharmaceutical ingredient, preventing premature or unintended exposure, diffusion, or contact between the encapsulated core and the external environment, matrix, or destabilizing agents under expected manufacturing, storage, and administration conditions.

The term “protective polymer coat” or “encapsulation coating” refers to one or more continuous or essentially continuous layers comprising one or more polymers applied to, formed around, or substantially enveloping at least a portion of an active pharmaceutical ingredient, thereby physically separating the ingredient from its external environment and imparting at least one of stabilization, controlled or targeted release, or taste- or odor-masking properties, within the oral dosage form.

The term “overcoating” refers to the application of an additional coating or layer over the surface of the finished dosage form—such as an oral thin film or gummy—intended to impart further barrier, stability, taste-masking, or handling characteristics, and which may be applied by dipping, spraying, pan coating, or other means.

The term “modified release” refers to any oral pharmaceutical dosage form or system in which the characteristics of drug release—such as onset, rate, duration, or site of release—are intentionally altered from those of an immediate release formulation. Modified release includes, but is not limited to, “extended release” (prolonged or sustained delivery to maintain therapeutic levels), “controlled release” (engineered to achieve a defined, reproducible release rate or profile), “delayed release” (initiation of release after a predetermined lag), “pulsatile release” (one or more rapid or discrete bursts interspersed with minimal release), “timed release” (programmed initiation or rate according to a schedule), “targeted release” (maximized delivery at a specific location), and other engineered release profiles, whether by formulation, coating, or encapsulation. The term “programmed release” is a subset wherein the release schedule, timing, or delivery pattern of the active ingredient is pre-determined or engineered, for example to align with circadian rhythms, therapeutic needs, or specific biological triggers

The term “loss of potency” refers to a measurable decline in the biological or chemical activity of the active pharmaceutical ingredient within the dosage form, determined by established analytical or pharmacopeial methods, such that the amount available falls below a specified percentage of the stated dose under tested storage, processing, or administration conditions.

The term “free-flowing” refers to a physical state wherein solid particulates, powders, or granules are capable of moving or pouring readily and uniformly, without significant aggregation, caking, or clumping, thereby facilitating uniform handling, mixing, or incorporation into formulations.

The term “uniform distribution” refers to a state in which an ingredient (such as the encapsulated active pharmaceutical ingredient) is substantially evenly dispersed throughout a matrix or medium, such that the concentration of the ingredient does not vary by more than a predefined, pharmaceutically acceptable margin across the dosage form.

The term “substantially isolated” refers to a condition wherein the encapsulated active ingredient is separated from its surrounding environment or destabilizing agents to an extent that substantially prevents degradation, interaction, or chemical exchange under intended manufacturing, storage, or administration conditions.

The term “stability” refers to the capacity of a composition, dosage form, or active ingredient to retain its original physical, chemical, biological, and/or functional properties within specified limits throughout its shelf life or under specified storage, handling, or use conditions.

The term “palatability” refers to the overall acceptance of a dosage form's taste, smell, texture, and mouthfeel by a subject upon administration, as assessed subjectively and/or in comparison to standard formulations.

The term “barrier function” refers to the ability of a coating, matrix, or encapsulation system to impede or substantially prevent the passage of water, acids, oxygen, light, or other destabilizing agents into contact with the encapsulated or embedded active ingredient, under standard processing, storage, or physiological conditions.

The term “functional agent” refers to any component of a dosage form, other than the primary active pharmaceutical ingredient, that provides a specific technical or auxiliary effect-such as modifying release, enhancing absorption, providing color or flavor, acting as a preservative, or imparting other properties important to product performance or acceptability.

The term “pharmaceutical grade” refers to materials or substances meeting the purity, safety, quality, and regulatory standards established for use in pharmaceutical products by recognized pharmacopeias or regulatory agencies.

The term “food grade” refers to materials or substances that meet applicable regulatory requirements for safety, purity, and use in products intended for human consumption, as established by relevant food safety authorities or regulations.

The term “pediatric, geriatric, or special-needs populations” refers to groups of human subjects who, due to age or specific health conditions (including difficulty swallowing, sensory impairments, or unique dietary restrictions), may require adapted dosage forms with modified size, flavor, dosage strength, or texture to facilitate safe and effective administration and improved compliance.

The term “absorption enhancer” refers to any substance co-administered or formulated with an active ingredient to increase the bioavailability or rate of absorption of the active ingredient across a biological membrane, such as the gastrointestinal mucosa.

The term “microparticle” refers to a discrete particulate entity, typically with a mean particle diameter in the range of 1 micron to 1000 microns (1 μm-1 mm), comprising a core of active substance and optionally one or more encapsulating layers or coatings.

The term “granule” refers to an aggregated solid particle with a mean diameter generally greater than 100 microns, formed by agglomeration, compression, or coating processes, and intended for use in pharmaceutical dosage forms as a delivery vehicle for active or inactive ingredients.

The term “coating thickness” refers to the average distance measured perpendicularly from the surface of the encapsulated material or core to the exterior surface of the surrounding polymer or coating layer, as determined by microscopy, particle analysis, or equivalent quantitative technique.

The term “encapsulation process” refers to any method, technique, or combination thereof by which a material—particularly an active pharmaceutical ingredient—is physically enveloped, entrapped, or surrounded by a coating, shell, or matrix, thereby producing a discrete, structurally coherent encapsulated form.

The term “burst release” refers to a release profile in which a significant portion of the encapsulated active ingredient is released rapidly after administration or upon exposure to triggering conditions, prior to the onset of any sustained or controlled release phase.

The term “programmed release” refers to a controlled drug-release strategy in which the timing, rate, or pattern of active ingredient release from the dosage form is pre-specified to achieve optimally timed pharmacodynamic effects, optionally aligned with biological rhythms or disease cycles.

The term “self-emulsifying formulation” refers to a composition that, upon exposure to an aqueous environment, spontaneously forms an emulsion or microemulsion without the need for high shear mixing, thereby enhancing solubility and absorption of hydrophobic active ingredients.

The term “staged release” refers to a release profile in which multiple, temporally or environmentally distinct phases of active ingredient release occur sequentially from the dosage form, with each phase being dictated by formulation or encapsulation design.

The term “enzyme-triggered release” refers to a release mechanism in which the encapsulation material, coating, or matrix surrounding an active pharmaceutical ingredient degrades, dissolves, or becomes permeable specifically in response to the activity of endogenous or exogenous enzymes, such as those in the digestive tract or produced by colonic bacteria. This strategy enables controlled and targeted release of actives in specific sites, such as the lower gastrointestinal tract, or upon exposure to certain metabolically relevant enzymes.

The term “analytical method” refers to any procedure or technology suitable for measuring, verifying, or characterizing the composition, structure, distribution, integrity, or performance characteristics of a pharmaceutical ingredient, encapsulation, or dosage form, including but not limited to spectroscopic, chromatographic, gravimetric, or microscopic means.

The term “quality control monitoring” refers to the application of analytical, statistical, or systematic examination of manufacturing parameters and product characteristics-such as coating integrity, particle size, encapsulation uniformity, or dose content-during or after production, in order to verify compliance with predetermined specifications.

The term “portion” refers to any measurable fraction, subset, or segment of a substance or composition, which may range from greater than 0% up to and including 100% of the total amount present, unless a specific quantitative value or range is otherwise specified.

The term “non-encapsulated” refers to an active ingredient or material that is not deliberately enclosed, coated, or surrounded by a continuous or intentional polymeric or protective barrier and is thus exposed to its surrounding environment within the dosage form.

The term “immediate release” refers to an oral dosage form, or a specific fraction (“immediate release portion”) of a dosage form, that is designed to rapidly disintegrate and/or make available substantially all of its active pharmaceutical ingredient for absorption or action within approximately 60 minutes following administration, without any intentionally engineered delay, sustained, or controlled release mechanism.

The term “multi-layer” (and “multi-component” or “dual-layer” when applicable) refers to an encapsulation architecture comprising two or more distinct concentric or sequential layers or compartments, each comprising the same or different materials or functionalities, arranged to provide additive or complementary protective, release, or targeting properties to the core ingredient.

The term “overcoat” or “overcoating” is an additional externally applied film or layer, distinct from the primary encapsulation, that is applied to the surface of a finished dosage form (e.g., gummy, oral film, or encapsulated particle) in order to impart further barrier, stability, water-resistance, oxygen-barrier, taste-masking, or physical protection properties, and can be implemented via dipping, spraying, pan coating, or other suitable methods.

The term “accelerated stability protocol” refers to a testing regimen in which a product or composition is subjected to stress conditions-such as elevated temperature, humidity, light, or other environmental factors—for a defined period in order to simulate or predict the stability, integrity, or shelf-life of the product under normal storage or use conditions.

The term “API potency” refers to the quantitative measurement of the biological, chemical, or pharmacological activity of the active pharmaceutical ingredient within the dosage form, relative to a defined reference standard or labeled content.

The term “edible polymer” refers to any natural, synthetic, or modified polymeric substance that is safe for oral ingestion, non-toxic at intended use levels, and compliant with food or pharmaceutical regulatory requirements for consumption by humans or animals.

The term “water-resistant” refers to a property or characteristic of a material, coating, or dosage form that limits or retards the ingress or transmission of water or moisture under intended storage, handling, or administration conditions.

The term “oxygen-barrier” refers to a material or layer that substantially inhibits or restricts the permeation or transfer of molecular oxygen from the surrounding environment into or through the treated surface or dosage form.

The term “unpleasant taste or odor” refers to any sensory characteristic of an ingredient or dosage form that is generally regarded as disagreeable, bitter, metallic, pungent, or otherwise non-palatable to a human or animal subject under typical use conditions.

The term “machine-readable marker” refers to any feature, tag, or embedded element on or within a dosage form, particle, or packaging that can be detected, scanned, or interpreted by an electronic device, including but not limited to radio-frequency identification tags (RFID), two-dimensional barcodes (such as QR codes), and digital adherence sensors, for the purposes of identification, tracking, or monitoring.

The term “bioavailability or absorption enhancer” refers to any co-administered agent, excipient, or formulation component that acts to increase the fraction or rate at which an active pharmaceutical ingredient becomes available at the site of physiological activity following administration.

The term “nanoparticle” or “nanocluster” refers to a discrete, particulate entity, aggregate, or structure with a mean dimension in the submicron scale, generally less than 1 micron and typically less than 100 nanometers, which may serve as a carrier for active ingredients, functional excipients, or as a structural or delivery component within pharmaceutical or nutraceutical dosage forms.

The term “encapsulation uniformity” refers to the consistency and evenness of the encapsulation process, as determined by the distribution, thickness, and integrity of the encapsulating layer(s) around the active ingredient particles throughout a batch or dosage form.

The term “multi-compartment dosage form” refers to a unitary oral dosage form comprising two or more physically distinct regions, layers, beads, or compartments, each containing at least one active ingredient, wherein at least one compartment is functionally or spatially separated from another either by encapsulation, matrix composition, or structural design.

The term “dual-layer encapsulation” refers to the encapsulation of an active pharmaceutical ingredient using two or more discrete, concentrically arranged coating layers, each comprising different or similar materials, and each independently selected to confer specific barriers or release properties.

The term “overcoat” refers to a supplementary, externally applied film or layer, different from the primary encapsulation, which encloses all or part of a dosage form for additional stability, moisture resistance, taste-masking, or physical protection.

The term “self-emulsifying formulation” refers to a composition containing surfactants and/or co-surfactants that rapidly form an oil-in-water emulsion or microemulsion upon contact with aqueous fluids, thereby enhancing the solubility and/or absorption of hydrophobic active ingredients.

The term “immediate and modified release portions” refers to a dosage form or ensemble in which at least part of the active ingredient is formulated for rapid onset (immediate release) and another part is formulated for delayed, sustained, targeted, or otherwise intentionally altered release compared to an unmodified matrix.

The term “smart-responsive polymer” refers to a polymer or polymer blend that undergoes a substantial and reversible alteration in physical or chemical properties—such as solubility, permeability, or integrity—in response to an internal or external stimulus, including but not limited to pH, temperature, enzyme presence, light, or magnetic field.

The term “bioadhesive excipient” refers to a pharmaceutical excipient that, when included in a formulation, increases the propensity of the matrix or encapsulated component to adhere to biological tissues, such as mucosal surfaces of the oral cavity or gastrointestinal tract, for a prolonged period.

The term “personalized or indication-specific modification” refers to any adaptation of the dosage form-such as adjustments in API amount, release kinetics, flavor, excipient profile, or physical characteristics-made to suit a particular patient group, disease state, or therapeutic requirement, as opposed to a universal formulation.

The term “chronotherapeutic release profile” refers to a release profile from a dosage form in which the timing, onset, rate, or pattern of drug release is intentionally programmed or engineered to coordinate with biological rhythms, disease symptoms, or external events.

The term “machine-readable code” refers to any data-encoded pattern, tag, or feature readable by an optical, electronic, or digital sensor or device for identification, authentication, or traceability purposes; including but not limited to barcodes, QR codes, RFID tags, and digital adherence sensors.

The term “co-encapsulation” refers to encapsulation of two or more active or functional ingredients together within the same polymeric or particulate casing or matrix, such that their release and protection are controlled concurrently by the encapsulant.

The term “hybrid encapsulation” refers to an encapsulation technology wherein two or more encapsulating materials—such as a blend of polymers and nanoparticles or a combination of natural and synthetic polymers—are employed in a single coating, layer, or structure to impart combined barrier and release characteristics not achievable with a single material alone.

The term “taste-sensing analytical technology” refers to a technology, methodology, or instrument that enables quantification, characterization, or analysis of taste or flavor attributes of a substance or dosage form, by either direct human sensory analysis or by electronic, chemical, or biochemical means.

The term “staged API release” refers to any drug delivery system in which two or more distinct phases of active ingredient release occur at pre-selected times, pH levels, or physiological locations after administration, such that the timing or site of API availability is temporally or spatially segmented.

### Oral Thin Films

A typical oral thin film (OTF) dosage form for delivery of encapsulated active pharmaceutical ingredients (APIs) is constructed from a combination of excipients chosen to provide the necessary mechanical integrity, flexibility, fast dissolution or mucoadhesion, stability, and palatability required for oral administration. The dosage form contains the encapsulated API dispersed within a polymeric film-forming matrix. Suitable film-formers include, for example, hydroxypropyl methylcellulose (HPMC), polyvinyl alcohol (PVA), sodium alginate, pullulan, hydroxypropyl cellulose (HPC), and methylcellulose. These are generally present in an amount ranging from about 10% to 60% by weight of the total film composition, with the exact amount determined by the desired thickness, strength, and dissolution profile of the final film. Plasticizers such as glycerin, propylene glycol, polyethylene glycol (e.g., PEG 400), or sorbitol are used to impart flexibility to the film, typically included at levels of about 2% to 10% by weight. The composition further includes water or an appropriate aqueous or hydroalcoholic solvent, generally in an amount of at least about 2% by weight, as a vehicle for creating the film-forming solution. For patient acceptability, one or more sweeteners such as sucralose, mannitol, stevia, xylitol, or aspartame may be present, with sweeteners typically ranging from 0.01% to 8% by weight depending on sweetness intensity and type. Taste-masking flavorants are commonly included, such as natural or artificial fruit flavors, mint, or vanilla, in amounts up to about 2% by weight. Acidulants or pH modifiers such as citric or tartaric acid may also be added at concentrations up to 1% to further improve taste and dissolution characteristics. Surfactants or wetting agents may be included to enhance dispersion of the encapsulated API within the matrix; common examples are polysorbate 80 and sodium lauryl sulfate, used at about 0.05% to 0.3% by weight. For visual appeal, colorants such as FD&C dyes, natural vegetable dyes, or titanium dioxide may be used in amounts ranging from 0.01% to 0.2%. Preservatives such as sodium benzoate or potassium sorbate may be optionally added at levels of about 0.05% to 0.2%, especially for multi-dose packaging or to extend shelf-life. Additional functional ingredients can include fillers (e.g., microcrystalline cellulose) at 1% to 10%, or antioxidants such as ascorbic acid, as needed for specific formulation stability or performance.

The manufacturing process for OTFs with encapsulated APIs typically begins with preparation of the encapsulated API itself, which consists of coating or entrapping the API with a protective polymer prior to introduction into the film slurry. Suitable polymers for encapsulation include HPMC, ethylcellulose, cellulose acetate phthalate (CAP), various Eudragit grades, chitosan, PEG, PVA, PMMA, PLA, alginate, or blends thereof. Encapsulation can be achieved using techniques such as spray drying, coacervation, solvent evaporation, or fluid bed processing to produce dry, free-flowing microparticles or granules with a controlled coating thickness, typically within the range of 10 to 200 microns. After encapsulation, the polymeric film-forming agents and plasticizers are dissolved in water with gentle heating and mixing to form a homogeneous aqueous or hydroalcoholic solution. Sweeteners, flavors, acidulants, surfactants, colorants, and any additional minor excipients are added sequentially under continued mixing. The encapsulated API is then carefully incorporated into the cooled film-forming solution, using low-shear stirring to ensure uniform distribution and avoid breaking the protective encapsulation. The resulting slurry is cast onto a non-stick backing, such as a silicone- or Teflon-coated tray or continuous belt, using a blade or slot-die applicator to control wet film thickness. The cast film is then dried by air, in a controlled-temperature oven (typically at 25-60° C.), or under reduced humidity to evaporate solvents, forming a flexible, solid film of desired uniform thickness, usually between 50 and 300 microns when dry. Once drying is complete, the film is cut into individual dosage units (typically between 2 and 20 square centimeters per unit), optionally embossed or perforated as needed for unit dosing. Each OTF is packaged, preferably in moisture- and oxygen-resistant material, to maintain stability of the API and protect against environmental degradation throughout storage and transport. Temperature, humidity, and mixing conditions are carefully controlled throughout the process to minimize the risk of premature release or degradation of the encapsulated API.

### Gummies

A typical gummy dosage form for delivery of an encapsulated API is constructed as a semi-solid matrix incorporating ingredients selected to achieve the desired texture, taste, stability, and controlled release characteristics for oral administration. The encapsulated API, coated or entrapped with a suitable protective polymer as described above, is uniformly dispersed within the gummy matrix. The matrix comprises one or more gelling agents such as gelatin, pectin, agar, or carrageenan, with gelatin typically used at 5% to 25% by weight, pectin at 2% to 8%, agar at 0.2% to 2%, and carrageenan at 0.1% to 1%. Water serves as the principal solvent and processing medium, present in an amount typically ranging from 2% to 8% by weight, facilitating dissolution or dispersion of gelling agents and solubilization of other excipients. Sweeteners or bulk carbohydrate sources such as sucrose, glucose syrup, corn syrup, fructose, stevia, and other sugars are included at high levels to provide palatability and appropriate mouthfeel, generally accounting for between 10% and 40% by weight. Acidulants including citric acid, malic acid, or tartaric acid may be included at about 0.2% to 2% to enhance taste and control pH. One or more flavoring agents—natural or artificial flavors—are incorporated in amounts of 0.1% to 2% to mask any bitterness or unpleasant API flavors. Emulsifiers and surfactants, such as lecithin, may be present at about 0.1% to 0.5% to ensure smooth texture and even dispersion of hydrophobic or encapsulated materials. Stabilizers or preservatives such as potassium sorbate or sodium benzoate can be added at about 0.05% to 0.2% by weight to extend shelf-life and prevent microbial growth. Colorants from either synthetic or natural sources, including FD&C dyes and fruit/vegetable extracts, are employed in amounts of about 0.01% to 0.5% for consumer appeal. Additional functional excipients may include fillers (modified starches) at 2% to 10%, plasticizers or humectants (e.g., glycerin, sorbitol) to modify chewiness, and antioxidants such as ascorbic acid at 0.05% to 0.5% where enhanced stability is required.

The manufacturing process for gummies with encapsulated APIs starts with preparation of the dry, coated API particles or granules using suitable encapsulation techniques as described above—such as spray drying, coacervation, solvent evaporation, or fluid bed coating—to achieve desired microcapsule size and coating properties. The gummy base is prepared by dissolving one or more gelling agents, sweeteners, and the required amount of water in a kettle or mixing vessel, then heating the mixture to temperatures typically between 70° C. and 95° C. to fully hydrate and dissolve the components and to form a clear, uniform slurry. When the gelling solution is homogeneous, the batch is cooled below about 60° C. to avoid damaging the integrity of encapsulated particles and to prevent the degradation of flavors, colors, or heat-sensitive preservatives. At this point, flavorings, acidulants, colors, stabilizers, and emulsifiers are thoroughly blended in, ensuring an even mixture. The encapsulated API, previously prepared and dried, is gently incorporated into the cooled slurry with low-shear mixing to ensure uniform distribution and to minimize risk of rupturing the coating. The gummy mixture is then poured or deposited into molds or onto trays, where it is allowed to cool and solidify, typically at ambient or refrigerated temperatures for 2 to 24 hours, depending on gel concentration and desired product texture. Once set, the gummies are removed from the molds, optionally dusted with a dry anti-caking agent such as cornstarch to prevent sticking, and packaged into moisture-inhibiting, light-protective containers to preserve both taste and potency. Throughout the process, careful control of mixing speed, temperature, and order of addition is maintained to maximize product uniformity and the effectiveness of the encapsulation, ensuring the API remains protected from destabilizing agents in the manufacturing environment and throughout the product's shelf life.

All excipients and functional agents included in any oral thin film (OTF) or gummy formulation, as described herein, are selected to be of pharmaceutical grade and/or food grade quality. Such excipients and functional agents are employed exclusively in amounts that are consistent with, or permitted by, the U.S. Food and Drug Administration (FDA) regulations and guidelines for use in pharmaceutical or dietary supplement products. Each excipient and functional agent are further present within established safety and regulatory limits, ensuring that the final dosage form meets all applicable standards for human consumption and adheres to current good manufacturing practices (cGMP) as recognized or required by the FDA.

### Encapsulation of API

Encapsulation of the active pharmaceutical ingredient (API) is accomplished through a controlled process designed to produce a protective barrier around at least a portion of the API, thereby physically isolating it from destabilizing agents in the semi-solid oral dosage form environment. The process begins by selecting a suitable encapsulating polymer based on the physicochemical properties of the API, the desired release characteristics, and the target site for release within the gastrointestinal tract. Representative polymers may include, but are not limited to, hydroxypropyl methylcellulose (HPMC), ethylcellulose, cellulose acetate phthalate (CAP), Eudragit grades, chitosan, polyvinyl alcohol (PVA), polyethylene glycol (PEG), polylactide (PLA), and blends thereof. The API is combined with the selected polymer or a polymer solution using a technique appropriate to the thermal, pH, and moisture sensitivity of the API, such as spray drying, coacervation, solvent evaporation, or fluid bed coating.

During spray drying, for example, a solution or suspension of the API and the desired polymer in a volatile solvent is atomized into heated air, causing rapid evaporation of the solvent and formation of discrete microcapsules or microparticles, each comprising an API core enveloped by a polymer shell. In coacervation, oppositely charged polymers or changes in pH and ionic strength are used to induce formation of a polymer-rich phase that deposits a coating around suspended API particles. Alternatively, solvent evaporation methods involve dispersing the API in a polymer-containing organic phase, emulsifying this mixture into an aqueous phase, and subsequently removing the solvent under carefully controlled conditions to solidify the polymer coat. In fluid bed coating, the API particles are suspended and fluidized in a stream of heated air while a solution or dispersion of the coating polymer is sprayed onto their surfaces; subsequent evaporation of solvent fixes the polymer shell around each individual API particle or granule.

The thickness, integrity, and functional properties of the encapsulation coating can be adjusted by varying process parameters such as polymer concentration, molecular weight, solvent composition, temperature, atomization conditions, and drying time, as well as by the use of additional stabilizers, plasticizers, or antioxidants in the encapsulation solution. This process yields free-flowing, dry microparticles or granules in which a substantial portion or all of the API is surrounded by a continuous polymer layer, typically between 10 and 300 microns in thickness, with some embodiments allowing for even thinner or thicker coatings as dictated by the intended release profile. The encapsulated API is then isolated, typically by filtration or centrifugation and subsequent drying, and is ready for incorporation into the semi-solid matrix, such as a gummy or oral thin film, under conditions that maintain the integrity of the protective coating and the chemical stability of the API throughout further processing, storage, and end use.

### Advanced Delivery Platforms

Modern drug delivery systems increasingly leverage technologies to maximize the uniformity, precision, and flexibility of drug release within semi-solid oral dosage forms such as gummies and oral thin films. In one embodiment, the semi-solid matrix may be incorporated into or contained within a secondary oral capsule, including a gelatin or plant-based capsule, providing enhanced dosing flexibility and secondary barrier protection, particularly for formulations bearing ultra-low or ultra-high doses of potent APIs. The capsule may be color-coded to minimize counterfeiting risks or enable batch tracking. Another embodiment utilizes micro-depositing technology, wherein APIs are precisely deposited onto the surface or within stratified layers of the oral thin film, enabling rapid absorption through buccal or sublingual delivery and highest available bioavailability for mucosal administration.

### Manufacturing Innovations and Stability Enhancements

Embodiments may include dosage forms manufactured by advanced processes, including but not limited to needle-free, high-voltage electrospinning, hot-melt extrusion, and 3D printing. These techniques enable the formulation of films from solution or suspension, support the incorporation of high API loading, and produce stable nanofibrous films with controlled drug release profiles and superior stability against crystallization or solvent-induced degradation. Additional embodiments utilize in-line imaging, spectroscopy, or automated quality-control monitoring during manufacture, ensuring the consistent thickness, uniformity, and potency of encapsulation or coated layers.

### Multi-Compartment and Combination Delivery

Commercial need for combination therapies has driven the invention of multi-compartment, multi-API dosage forms. In these embodiments, the oral dosage form may consist of two or more APIs, each independently encapsulated, co-encapsulated, or distributed in both immediate and sustained-release formats within the same dosage or within internal dual-layer or compartmentalized architecture. Biphasic release may be achieved by suspending both IR and ER microparticles in a liquid or matrix phase, or by encapsulating separate tablet or bead cores within the same dosage unit. Embodiments may include immediate release in one portion and extended release in another, aiding both initial onset and maintenance of therapeutic effect.

### Nanotechnology and Bioavailability Enhancements

Embodiments using nanotechnology focus on increasing API solubility and enhancing oral bioavailability. Integrating nanoparticles or nanoclusters into the matrix, encapsulation, or as API carriers can reduce dosage requirements, minimize side effects, and improve systemic absorption by avoiding first-pass metabolism. Furthermore, rapid dissolution and absorption are achieved by creating ultra-thin films or nanofiber structures, while surface-modifying particles with bioadhesive ligands increases mucosal retention and local bioavailability.

### Microencapsulation and Taste Masking Technologies

Microencapsulation by coacervation or spray congealing, and creation of uniformly dimensioned microspheres, offer highly efficient taste masking, variable release kinetics, and enable the creation of customized dosage forms for patient-specific populations. Coatings may use semi-permeable polymers down to microscale thickness, physically isolating bitter-tasting APIs and ensuring consistent masking. Proprietary taste-masking processes may enable rapid production, variable particle shapes, and multiparticulate architectures suitable for chewable, dissolvable, or food-like textures.

### Regulatory, Sensory, and Consumer-Oriented Embodiments

Embodiments tailored for specific populations (pediatric, geriatric, special-needs) include the use of sensory-optimized flavors, tailored matrix textures, and allergen-free excipient systems, ensuring broad compatibility with dietary and regulatory requirements. These may further employ validated taste sensing analytical technologies and GRAS-compliant materials for global regulatory acceptance. Other consumer-oriented innovations include single-dose packaging, dose titration features, digital adherence sensors, and inclusion of anti-counterfeiting markers or machine-readable codes embedded within the dosage form.

### Precision Dosing and Rapid Film Technologies

Innovations may comprise rapid-dissolving films designed to release a full therapeutic dose within seconds of administration, ensuring optimal onset for APIs that require fast pharmacodynamic activity. Films developed by solvent casting or hot-melt extrusion can further be tailored for kinder sensory properties (mouthfeel, texture) and maximize dose uniformity in large-scale production.

### Biologics, Probiotics, and Enzyme-Triggered Release

Embodiments disclosed herein include platforms suitable for biologics, gene therapies, and probiotics which require unique protection and activation profiles. Matrix and encapsulation systems may be formulated to employ enzyme-triggered release, such that microbial or digestive enzymes in the lower GI tract initiate the release of live cells, proteins, or RNA APIs, optimizing delivery and viability for functional therapies.

### Reducing Agent and API Stability Additives

Stability solutions may involve incorporating reducing agents within the polymer matrix or encapsulation to prevent chemical oxidation or degradation of sensitive APIs through the oral thin film or gummy environment. Additives such as glutathione, dithiothreitol, or ascorbate can be embedded at measured concentrations, significantly extending the shelf-life and functional integrity of administered compounds.

### Smart-Responsive Polymers and Triggered Release Systems

In certain embodiments, the encapsulation of the active pharmaceutical ingredient (API) may utilize smart polymers that respond to specific physiological stimuli such as temperature, pH, or enzymatic activity within the gastrointestinal tract. These smart-responsive polymers enable real-time adaptation of drug release, so that the encapsulated API is released only upon detection of precise physiological triggers, such as a shift from gastric to intestinal pH, or in response to bacterial enzymes present uniquely in the colon. For example, thermoresponsive polymers may be included to provide temperature-triggered dissolution for site-specific or time-dependent delivery, while enzyme-cleavable linkers incorporated into the encapsulation matrix may allow for release only in the presence of colonic microflora or digestive enzymes, thereby achieving highly targeted and efficient delivery for drugs or biologics intended for local action or improved systemic absorption.

### Microstructure and Texture Optimization for Compliance

To enhance commercial acceptability and patient adherence—especially among pediatric and geriatric populations—an embodiment incorporates precise control of semi-solid matrix texture via optimization of multiple micromechanical parameters. The formulation process includes measuring and adjusting physical characteristics such as adhesiveness, chewiness, cohesiveness, firmness, hardness, springiness, and resilience within pre-specified ranges. The resultant oral dosage forms deliver a consistent, reproducible texture profile (e.g., a specific chewiness or resilience), improving both the organoleptic experience and uniformity of drug delivery. Moreover, intraoral dissolution pH, weight uniformity, and moisture content are carefully controlled and specified, ensuring both regulatory compliance and consumer preference.

### Film and Gummy Technologies for Prolonged or Mucoadhesive Delivery

Further embodiments utilize mucoadhesive film or gummy matrices that prolong the residence time of the dosage form in the oral cavity or on mucosal surfaces. By incorporating mucoadhesive excipients or structuring the matrix for increased oral cavity adherence, the dissolution rate and drug absorption window are extended, supporting indications such as local mouth/throat delivery, chronic conditions, or therapies requiring extended mucosal contact. For example, thin films containing mucoadhesive agents enable gradual release of actives for oral care, pain management, or anti-infective applications, delivering benefits of increased efficacy and improved patient compliance.

### Fast-Dissolving and Multi-Functional Film Innovations

Some embodiments employ rapid-dissolving film technology constructed from combinations such as polyvinyl alcohol, polyethylene glycol, rice starch, and pH-adjusting agents. These films can robustly encapsulate unstable APIs by incorporating reducing agents such as glutathione, methionine, or dithiothreitol within the matrix to dramatically inhibit oxidative or hydrolytic degradation. Additionally, such films may include essential oils or natural antimicrobials for multi-functional benefit, such as simultaneous drug delivery, oral care, and breath freshening. The manufacturing process may further leverage bottom-side substrate drying to achieve remarkably low non-uniformity and moisture, supporting high-value stability and bioavailability benefits for commercial products.

Modified Release Capsules with Multi-Compartment Semi-Solid Fills

In still further embodiments, the semi-solid oral dosage form may be manufactured as a multi-compartment or multi-particulate system, including the placement of semi-solid or gelled API-containing matrices within a hard or soft capsule shell or as separate beads inside a larger softgel. These capsules may be specially engineered to provide controlled, timed, or dual-phase release by combining different encapsulation or granulation techniques within a single container.

Softgel forms may specifically encapsulate oil-based, lipophilic, or combination APIs, maximizing dosing accuracy and protecting moisture-sensitive contents via seamless, hermetically sealed shell construction. Additionally, beadlets with unique coatings for sequential or sustained release may be suspended within a semi-solid or gelled base inside the capsule, offering highly programmable release profiles.

### Sugar-Free, Vegan, and Customizable Gummy Platforms

A further embodiment enables the use of sugar-free sweeteners (such as sorbitol or xylitol) and plant-based gelling agents (such as pectin or agar) to produce vegan, low-glycemic, and allergen-free gummies. The formulation provides equivalent or improved texture, taste, and chew compared to traditional gelatin- and sucrose-based products, opening new market opportunities for health-conscious or restricted-diet populations. Customizable flavor, color, or shape features—using natural or synthetic colorants and flavoring agents—enable branding flexibility and the ability to tailor gummies for specific age groups, indications, or patient-prescriber markets.

### Embedded Quality Control, Traceability, and Anti-Counterfeiting

To address regulatory and commercial requirements, one embodiment integrates embedded batch identification or traceability markers into the matrix-such as edible QR codes, color-shifting pigments, or molecular tags-facilitating digital quality control, authentication, and anti-counterfeiting efforts. These features empower closed-loop supply chain verification, prescription compliance monitoring, and faster batch recall, while upholding product integrity and patient safety.

### Multi-API and Multi-Functional Delivery

Multi-functional embodiments deliver more than one API, nutritional, or cosmetic agent within the same matrix or as separate, segregated zones or layers within a film or gummy dosage form. This approach supports fixed-dose combination therapy, addition of supplement, vitamin, or probiotic adjuncts, or synergistic API/fluoride/antimicrobial pairings for oral healthcare. Such multi-compartment matrices can be engineered for independent or staged API release, further broadening therapeutic utility and market flexibility.

### Environmental Sustainability and Biodegradable Packaging

The semi-solid oral dosage forms, including gummies and oral thin films, can be manufactured using environmentally sustainable practices and materials. In certain embodiments, the matrix, encapsulating polymers, or packaging are selected to be biodegradable, compostable, recyclable, or derived from renewable resources. This includes, but is not limited to, using polylactic acid (PLA), polyhydroxyalkanoates, or microbial exopolysaccharides for encapsulation, and plant-based biopolymers or paper composites for exterior packaging. Such innovations minimize environmental impact by supporting safe disposal after use and compliance with green pharmaceutical initiatives. Furthermore, combination of biodegradable polymers with oxygen- and water-barrier properties ensures that reducing environmental footprint does not compromise product stability or shelf-life.

### Dose Titration, Patient Splitting, and Customization

The invention further enables dose titration and patient-personalized administration by providing dosage units that are subdividable, scored, or perforated. This facilitates accurate splitting by caregivers or patients for finer titration in pediatric, geriatric, or pharmacogenomically tailored regimens, and supports reduced dosing errors or improved adherence. Customizable formulations, such as those adjustable in active pharmaceutical ingredient content, flavor, color, or texture, may be produced via 3D printing or batch-down mixing strategies, allowing rapid adaptation for individualized patient needs, special indications, or market-driven preferences. These features are especially advantageous for populations requiring frequent dose adjustments, such as those undergoing chronic therapy or pediatric growth phase transitions.

### Inclusion of Imaging Agents and Diagnostic Tracers

Select embodiments may incorporate imaging agents, dyes, or diagnostic tracers into the encapsulation or dosage form matrix, allowing non-invasive confirmation of gastrointestinal (GI) transit, product location, or proper administration. The imaging agent can be selected from compounds or nanoparticles providing contrast in X-ray, MRI, or optical imaging, and may be embedded in the encapsulation layer, matrix, or as a separate compartment. This capability enables dual therapeutic and diagnostic (theranostic) utility, supports clinical studies of swallowing and dissolution behavior, and can provide adherence monitoring or confirmation of site-specific drug release for drugs requiring localized action in the GI tract.

Combination with Medical Devices and Digital Health Integration

In further embodiments, the dosage form is designed for compatibility with electronic medication management systems, drug delivery trackers, or adherence-monitoring devices. This includes compatibility with smart blister packs, ingestible digital sensors, RFID tracking tags, or packaging designed for automated medication dispensing. Integration with digital health infrastructure enables real-time tracking, patient reminders, remote adherence verification, and data generation for clinical or regulatory purposes. The dosage form may contain embedded, machine-readable codes or sensors for batch-level or unit-level traceability, supporting secure supply chains, prescription compliance, and efficient recall procedures if needed.

### Applications in Veterinary, Pediatric, and Special Needs Populations

The invention encompasses dosage forms specifically adapted for non-human animal health, pediatric, geriatric, and dysphagic populations. Formulation choices such as excipient selection, active ingredient load, and flavor profiles can be tailored for species-specific or age-specific acceptability, nutritional needs, safety, and regulatory requirements. For veterinary use, dosage forms may utilize flavors and matrices suitable for canine, feline, or other animal palatability and compliance, as well as encapsulation systems robust to non-human oral environments. Likewise, pediatric formulations may employ child-friendly sizes, chewability, and flavors, minimizing dosing aversion and maximizing medication adherence across diverse patient populations.

### On-Demand, Continuous, and Automated Manufacturing

In some embodiments, manufacturing processes for the described oral dosage forms are engineered for high-throughput, on-demand, or continuous automated production. This includes application of twin-screw extrusion, microfluidic encapsulation, 3D printing, or in-line spectroscopy for real-time monitoring of encapsulation integrity, uniformity, and active ingredient dose. These approaches enable rapid scaling, customization, and quality assurance compliant with current good manufacturing practice (cGMP), and support the development of adaptive manufacturing lines for precision medicine, clinical trials, or emergent market needs.

### Analytical and In-Process Verification Technologies

In certain embodiments, the invention provides for real-time and post-process analytical verification of the encapsulation, matrix, and dosage form characteristics. Quality assurance may include on-line or in-line spectroscopy, such as near-infrared (NIR), Raman, or ultraviolet-visible (UV-Vis) spectroscopy, as well as microscopic imaging (e.g., scanning electron microscopy, confocal microscopy, or laser diffraction-based particle sizing), to verify coating thickness, encapsulation integrity, and API distribution throughout the manufacturing process. The system may further integrate tracer diffusion studies, permeability testing, and fluorescent or dye-based labeling of APIs or excipients to non-destructively assess isolation and uniformity of encapsulation. These integrated analytical capabilities ensure batch-to-batch consistency, regulatory compliance, and product safety throughout continuous or batch manufacturing.

### Multi-Layer Encapsulation for Orthogonal Protection and Release

The dosage form can comprise a multi-layer (multi-shell) encapsulation system, where each discrete layer offers a complementary protection and/or release functionality. For example, an inner layer may consist of a time-dependent or enzymatically degradable polymer to delay exposure to gastrointestinal conditions, while an outer layer utilizes a pH-responsive or water-barrier material for site-specific release. Each layer can be independently formulated to provide orthogonal barrier properties (e.g., oxygen/moisture/acid/UV) or release triggers, supporting highly customizable therapeutic regimens and strengthening protection for extremely labile APIs or biologics. This approach also permits the inclusion of functional agents, such as mucoadhesive moieties, absorption enhancers, or flavor-masking agents, in one or more discrete layers surrounding the API core.

### Co-Encapsulation and Hybrid Particle Systems

The invention also encompasses co-encapsulation of multiple actives, functional agents, or excipients within the same polymeric or composite microcapsule or granule. Co-encapsulated systems may combine two or more APIs that require synchronized or synergistic release, or pair an API with solubility, permeability, or stability enhancers. Hybrid particle systems may blend natural and synthetic polymers, inorganic and organic materials, or combine nanoparticles with microparticles within a single granule, thereby offering tailored release profiles and protective characteristics not achievable with monolithic systems. Such multi-agent encapsulation supports combination therapies, compliance with complex dosing protocols, and the delivery of otherwise incompatible actives in a single, stable, and palatable dosage form.

### Personalized or Indication-Specific Formulation Adaptation

In various embodiments, the semi-solid dosage forms may be dynamically adjusted or customized based on patient-specific data, indication, pharmacogenomics, or disease state. The system may leverage modular encapsulation techniques, real-time mixing, or 3D printing to vary dose, release profile, API combination, excipient selection, flavor, color, or texture on a case-by-case basis. This facilitates the development of highly targeted therapies, pediatric or geriatric customization, and rapid adaptation for rare diseases, unique side-effect profiles, or market-driven preferences. Such formulations are especially advantageous in direct-to-patient, compounding, or decentralized manufacturing environments, and are supported by the system's analytical and flexible production capabilities.

### Packaging for Enhanced Stability and Compliance

Select embodiments include specifically engineered packaging solutions that further enhance stability, patient compliance, and regulatory traceability. Package designs may include compartmentalization for multi-dose regimens, moisture- and oxygen-barrier layers, child-resistant features, single-dose or multi-dose blister packs, and embedded or printed machine-readable authenticity codes. Integration with digital health management systems allows for remote monitoring and inventory tracking, thereby supporting clinical trials, home healthcare, and institutional pharmacy distribution. Such packaging systems are designed to function as an essential complement to the stability and performance of the encapsulated semi-solid dosage forms.

### Specific Ranges, Values, and Embodiments

The specific embodiments describing the subject matter, ranges, and values provided below are for illustration purposes only, and do not otherwise limit the scope of the disclosed subject matter, as defined by the claims.

In specific embodiments, the semi-solid oral dosage form is an oral thin film (OTF) or gummy.

In specific embodiments, the semi-solid oral dosage form contains at least 2 wt. % water.

In specific embodiments, the semi-solid oral dosage form contains at least 3 wt. % water.

In specific embodiments, the semi-solid oral dosage form contains at least 4 wt. % water.

In specific embodiments, the semi-solid oral dosage form contains at least 5 wt. % water.

In specific embodiments, the semi-solid oral dosage form contains at least 6 wt. % water.

In specific embodiments, the semi-solid oral dosage form contains at least 8 wt. % water.

In specific embodiments, the polymer comprises a pH sensitive polymer.

In specific embodiments, the polymer dissolves at pH above 6.0, to release the API in the intestine.

In specific embodiments, the polymer dissolves at pH above 7.0, to release the API in the colon.

In specific embodiments, the polymer comprises a cellulose derivative.

In specific embodiments, the polymer comprises methacrylic acid copolymers.

In specific embodiments, the polymer comprises a biodegradable polymer.

In specific embodiments, the polymer comprises a synthetic polymer.

In specific embodiments, the polymer comprises a time dependent polymer.

In specific embodiments, the polymer degrades in the presence of enzymes in the lower gastrointestinal tract.

In specific embodiments, the polymer comprises at least one of: Hydroxypropyl methylcellulose (HPMC); Ethylcellulose; Cellulose acetate phthalate (CAP); Eudragit L100; Eudragit S100; Eudragit FS30D; Shellac; Polyvinyl acetate phthalate (PVAP); Chitosan and its derivatives; Pectin; Guar gum; Xanthan gum; Polyvinylpyrrolidone (PVP); Polyethylene glycol (PEG); Hydroxypropyl cellulose (HPC); Polyvinyl alcohol (PVA); Poly(acrylic acid) (PAA); Poly(methacrylic acid-co-ethyl acrylate); Gelatin (used in combination with other polymers); Poly(lactic acid) (PLA); Poly(lactic-co-glycolic acid) (PLGA); Poly(ε-caprolactone) (PCL); Alginate; Carbopol (polyacrylic acid); Gellan gum; Poly(β-amino ester); Polyvinylacetal diethylaminoacetate (AEA); Eudragit RS and/or RL; Poly(methyl methacrylate) (PMMA); Hyaluronic acid; Carboxymethyl cellulose (CMC); and Polyurethanes.

In specific embodiments, the encapsulated active pharmaceutical ingredient (API) is characterized by analytical methods such as scanning electron microscopy, confocal fluorescence microscopy, particle size analysis, Fourier-transform infrared spectroscopy, or similar techniques to confirm coating thickness, integrity, and absence of direct contact with matrix excipients.

In specific embodiments, the encapsulation coating is designed to prevent significant transfer or diffusion of water, acids, or excipients to the API core, as confirmed by permeability or barrier testing.

In specific embodiments, the dosage form contains two or more APIs, wherein the APIs may be co-encapsulated within a single particle or separately encapsulated, optionally using polymers with different release or protection characteristics.

In specific embodiments, a dosage form combines immediate release and modified release APIs, achieved by including a portion of unencapsulated API and a portion that is encapsulated for delayed, controlled, or extended release.

In specific embodiments, the API comprises a peptide, biologic, RNA, mRNA, or gene therapeutic agent, and the encapsulation is adapted to maintain biological activity and stability under manufacturing and storage conditions.

In specific embodiments, the encapsulation comprises less-common or novel polymers, such as thiolated polymers, functionalized polysaccharides, photosensitive, or redox-responsive polymers.

In specific embodiments, the encapsulation comprises a blend of at least two polymers chosen to optimize both protection and release profile, such as combining an enteric polymer with a time-dependent polymer.

In specific embodiments, the encapsulation comprises nanomaterials, lipid excipients, surfactants, or other additives to enhance barrier function, dispersibility, or bioavailability.

In specific embodiments, the encapsulation shell is functionalized with ligands, antibodies, or lectins for targeted delivery or mucoadhesion in the gastrointestinal tract.

In specific embodiments, the encapsulating polymer is cross-linked by an in situ reaction, such as UV curing or thermal cross-linking, to enhance coating robustness.

In specific embodiments, encapsulation and/or dosage form manufacture is performed by continuous or automated processing, including techniques such as twin-screw extrusion, microfluidics, or robotic mixing.

In specific embodiments, the encapsulated API is released in response to an external stimulus, such as light, ultrasound, or magnetism, in addition to pH, enzyme, or time triggers.

In specific embodiments, the encapsulation comprises a self-emulsifying or self-dispersing formulation to improve solubility and absorption upon release.

In specific embodiments, the encapsulation comprises multiple concentric layers or compartments, supporting stepwise, pulsatile, or sequential release of one or multiple APIs.

In specific embodiments, the encapsulation comprises one or more taste- or odor-masking layers, optionally including colorants for improved patient acceptability or identification.

In specific embodiments, the entire oral thin film or gummy dosage form is further coated after final manufacturing with a water-resistant, oxygen-barrier, or taste-masking film.

In specific embodiments, the dosage form comprises an embedded marker, tracking feature (RFID or QR code), or digital adherence sensor.

In specific embodiments, the composition demonstrates, or is expected to demonstrate, improved pharmacokinetic or pharmacodynamic profiles over non-encapsulated reference formulations, as determined by in vitro gastrointestinal simulation, dissolution, or artificial gut models.

In specific embodiments, the dosage form comprises an immunogenic API and encapsulation modulates or improves the immune response elicited by the API.

In specific embodiments, the dosage form is adapted for pediatric, geriatric, or special-needs populations, providing easy-to-swallow, fast-dissolving, or specially flavored variants to enhance compliance.

In specific embodiments, the semi-solid oral dosage form is intended for animal health or veterinary use, and is formulated with excipients and flavors suitable for non-human mammals.

In specific embodiments, the encapsulated API and/or the finished dosage form demonstrate stability when subjected to conditions recommended in ICH guidelines for accelerated and long-term storage, maintaining specification for at least 12, 24, or 36 months.

In specific embodiments, the encapsulation coating is characterized by scanning electron microscopy, atomic force microscopy, or confocal microscopy to confirm uniformity, thickness, or integrity of encapsulation.

In specific embodiments, the encapsulated API is identified as being substantially isolated from water, acid, oxygen, or other destabilizing agents by permeability testing or tracer diffusion studies.

In specific embodiments, the semi-solid oral dosage form comprises two or more active pharmaceutical ingredients, wherein each API may be encapsulated independently, co-encapsulated, or left unencapsulated as desired.

In specific embodiments, the dosage form comprises an active ingredient selected from nucleic acids, peptides, proteins, monoclonal antibodies, vaccines, or gene therapies.

In specific embodiments, the encapsulation comprises a blend of natural and synthetic polymers, including but not limited to polysaccharide derivatives, polyanhydrides, polyesters, or thiolated polymers.

In specific embodiments, the encapsulation incorporates functional groups for mucoadhesion, targeting, or cellular uptake, such as lectins, folate, cell-penetrating peptides, or antibodies.

In specific embodiments, the encapsulation includes nanoparticles, microparticles, or lipid-based additives to enhance barrier properties or enable responsive release profiles.

In specific embodiments, the dosage form comprises a multilayered encapsulation, wherein each discrete layer provides protection or release in response to a distinct environmental trigger (e.g., pH, enzyme, time, or mechanical force).

In specific embodiments, the entire semi-solid dosage form (e.g., the finished gummy or OTF) is overcoated with an edible polymer film for improved handling, barrier properties, or packaging compatibility.

In specific embodiments, the encapsulation or matrix is formed by advanced manufacturing processes, including spray congealing, supercritical fluid encapsulation, microfluidic droplet generation, or 3D printing.

In specific embodiments, the encapsulation polymer is cured or crosslinked in situ by photoinitiated, chemical, thermal, or enzymatic crosslinking mechanisms.

In specific embodiments, the encapsulation is designed for API release triggered by a physical external stimulus selected from heat, light, magnetic field, ultrasound, or electrical stimulation.

In specific embodiments, the encapsulation system provides for simultaneous or staged release of multiple APIs, or pulsatile release according to a pre-selected pharmacokinetic schedule.

In specific embodiments, the encapsulation comprises a flavor-masking, sweetening, or aroma-masking outer layer composed of generally recognized as safe (GRAS) food additives.

In specific embodiments, the dosage form includes indicators, tracers, markers, or embedded information for patient or provider use (e.g., digital adherence sensors, RFID tags, or QR codes).

In specific embodiments, the composition is formulated for pediatric, geriatric, dysphagic, or veterinary patient populations and includes size, flavor, or texture adaptations to improve compliance.

In specific embodiments, the composition, process, and packaging are aligned with ICH or FDA stability, safety, and labeling requirements, including enhanced data supports for shelf-life, lot-to-lot uniformity, or child-resistance features.

In specific embodiments, the encapsulation enables the API to remain chemically and physically stable during high-humidity or wide temperature excursions as encountered in real-world storage, transit, or administration (>90% humidity, −20° C. to 60° C.).

In specific embodiments, the encapsulation is designed to withstand aggressive processing conditions, such as high-shear mixing, high-speed molding, or prolonged thermal exposure, without compromising integrity.

In specific embodiments, the dosage form includes both encapsulated and non-encapsulated ingredients to provide a combination of immediate and modified release pharmacokinetics.

In specific embodiments, the method of manufacture comprises a step of real-time or automated quality control monitoring of coating integrity, particle size, or uniformity.

In specific embodiments, the particles comprising the encapsulated API are characterized by mean particle size, particle size distribution, or zeta potential, as measured by dynamic light scattering, laser diffraction, or equivalent analytical methods.

In specific embodiments, the encapsulation coating is analyzed by differential scanning calorimetry, thermogravimetric analysis, or X-ray diffraction to confirm polymer composition, phase, or barrier function.

In specific embodiments, the dosage form, encapsulation, or API includes a batch-specific, machine-readable or visual marker to enable digital traceability, quality control, or anti-counterfeit measures.

In specific embodiments, the encapsulating polymer comprises a biodegradable, biosourced, or renewable material, such as polylactic acid, polyhydroxyalkanoate, or microbial exopolysaccharides.

In specific embodiments, the encapsulation includes a polymer or blend that swells but does not fully dissolve in biological fluid, providing controlled swelling-mediated release.

In specific embodiments, the excipients in the matrix include bioadhesive, mucoadhesive, or mucopenetrating agents such as carbomer, chitosan, thiolated chitosan, or polycarbophil.

In specific embodiments, the oral thin film or gummy matrix incorporates prebiotics, vitamins, minerals, or additional nutraceuticals in either free or encapsulated form.

In specific embodiments, the encapsulated API is manufactured by a process including a step of freeze drying or lyophilization to enhance stability.

In specific embodiments, the method of manufacture comprises a quality assurance step using in-line imaging or spectroscopy to verify encapsulation uniformity or API potency.

In specific embodiments, the encapsulated API is formed in a continuous manufacturing process, such as hot-melt extrusion or microfluidic encapsulation.

In specific embodiments, APIs are encapsulated at the nanoscale, with average encapsulated particle size less than 1000 nm, to enhance oral absorption or dissolution.

In specific embodiments, the encapsulation is designed to release the API in response to a change in ionic strength, redox potential, or the presence of specific gastrointestinal enzymes or bacteria.

In specific embodiments, the encapsulation achieves a programmed, stepwise, or multiphase release profile by varying the properties, thickness, or combinations of encapsulation materials.

In specific embodiments, the encapsulation restricts or delays release until a specific period or event post-ingestion, simulating chronotherapeutic or circadian dosing needs.

In specific embodiments, the product is adapted for use in dose titration or split-dosing applications by being scored, perforated, or otherwise subdividable by the end user or caregiver.

In specific embodiments, the semi-solid dosage form is formulated to have a specific shape, color, or texture for improved recognition, handling, or appeal to particular patient populations including children or geriatric subjects.

In specific embodiments, the dosage form is free from one or more major allergens or animal-derived ingredients, and is suitable for vegan, kosher, or halal applications.

In specific embodiments, the oral dosage form is provided in single-dose, multi-dose, or multi-compartment packaging designed to minimize moisture and oxygen exposure upon repeated access or use.

In specific embodiments, the matrix, encapsulating polymers, or packaging are designed to be biodegradable, compostable, or suitable for green disposal without environmental hazard.

In specific embodiments, the composition, method of manufacture, and testing protocols meet or exceed regulatory safety and stability requirements of the FDA, EMA, ICH, and/or other applicable regulatory authorities.

In specific embodiments, the oral dosage form is compatible with existing regulatory monographs for both pharmaceutical drugs and dietary supplement products.

In specific embodiments, the dosage form comprises more than one API, each with independently optimized encapsulation or release profiles, facilitating dual or multi-drug therapy.

In specific embodiments, the matrix or encapsulation includes personalized, patient-specific, or indication-specific modifications to enhance efficacy or minimize side effects, such as allergen-avoidance, flavor adaptation, or pharmacogenomic matching.

In specific embodiments, the dosage form is designed for use in combination with a medical device, such as a drug delivery tracker, blister pack detector, or electronic medication management system.

In specific embodiments, the composition is manufactured, packaged, and labeled according to cGMP and FDA or EMA requirements, with the encapsulation enabling compliance with regulatory standards for stability, dosing consistency, and safety.

In specific embodiments, all of the API is encapsulated.

In specific embodiments, a portion of the API is encapsulated.

In specific embodiments, a portion of the API is encapsulated, such that the portion that is not encapsulated is available for immediate release and the portion that is encapsulated is available for extended release, controlled release, sustained release, modified release, delayed release, pulsatile release, or timed release.

In specific embodiments, at least a portion of the API is encapsulated, such that the active ingredient that is encapsulated does not come in direct contact with the one or more excipients.

In specific embodiments, at least a portion of the API is encapsulated, such that the active ingredient that is encapsulated does not come in direct contact with water or acidifying agent.

In specific embodiments, the polymer encapsulates the API, such that the coating has an average thickness of up to 750 μm.

In specific embodiments, the polymer encapsulates the API, such that the coating has an average thickness of up to 500 μm.

In specific embodiments, the polymer encapsulates the API, such that the coating has an average thickness of up to 400 μm.

In specific embodiments, the polymer encapsulates the API, such that the coating has an average thickness of up to 300 μm.

In specific embodiments, the polymer encapsulates the API, such that the coating has an average thickness of up to 200 μm.

In specific embodiments, the polymer encapsulates the API, such that the coating has an average thickness of at least 1 μm.

In specific embodiments, the polymer encapsulates the API, such that the coating has an average thickness of at least 5 μm.

In specific embodiments, the polymer encapsulates the API, such that the coating has an average thickness of at least 10 μm.

In specific embodiments, the polymer encapsulates the API, such that the coating has an average thickness of at least 15 μm.

In specific embodiments, the polymer encapsulates the API, such that the coating has an average thickness of at least 20 μm.

In specific embodiments, the polymer encapsulates the API, such that the coating has an average thickness of at least 25 μm.

In specific embodiments, the polymer encapsulates the API, such that the coating has an average thickness of at least 50 μm.

In specific embodiments, the polymer encapsulates the API, for controlled release of the API.

In specific embodiments, the polymer encapsulates the API, for pulsatile release of the API.

In specific embodiments, the polymer encapsulates the API, for targeted release of the API.

In specific embodiments, the polymer encapsulates the API, for sustained release of the API.

In specific embodiments, the polymer encapsulates the API, for timed release of the API.

In specific embodiments, the polymer encapsulates the API, for prolonged release of the API.

In specific embodiments, the polymer encapsulates the API, for pH dependent release of the API.

In specific embodiments, the polymer encapsulates the API, for enzyme triggered release of the API.

In specific embodiments, the polymer encapsulates the API, for extended release of the API.

In specific embodiments, the polymer encapsulates the API, for delayed release of the API.

In specific embodiments, the polymer encapsulates the API, for modified release of the API.

In specific embodiments, the polymer encapsulates the API, for release of the API in the lower GI tract.

In specific embodiments, the polymer encapsulates the API, for release of the API in the lower intestine.

In specific embodiments, the polymer encapsulates the API, for release of the API in the colon.

In specific embodiments, the API is poorly water soluble.

In specific embodiments, the API has a low oral bioavailability (BA).

In specific embodiments, the API degrades in presence of oxygen.

In specific embodiments, the API degrades in presence of acid (pH<7).

In specific embodiments, the API degrades in presence of acid (pH<6.5).

In specific embodiments, the API degrades in presence of water.

In specific embodiments, the API degrades in presence of UV light.

In specific embodiments, the API has one or more functional groups that degrade in the presence of water.

In specific embodiments, the API has an unpleasant taste or flavor.

In specific embodiments, the water sensitive API includes one or more of desmopressin (DDAVP) (D-amino D-arginine vasopressin); dronabinol ((−)-trans-Δ⁹-tetrahydrocannabinol); aspirin (acetylsalicylic acid); penicillin (PCN); dipyridamole; vorapaxar; procaine; atorvastatin; azithromycin; pseudoephedrine; tiagabine; acitretin; rescinnamine; lovastatin; tretinoin; isotretinoin; simvastatin; ivermectin; verapamil; oxybutynin; hydroxyurea; selegiline; esterified estrogens; tranylcypromine; carbamazepine; ticlopidine; methyldopahydro; chlorothiazide; methyldopamine; naproxen; acetaminophen; erythromycin; bupropion; rifapentine; penicillamine; mexiletine; verapamil; diltiazem; ibuprofen; cyclosporine; saquinavir; morphine; sertraline; cetirizine; N-[[2-methoxy-5-(1-methyl)phenyl]methyl]-2-(diphenylmethyl)-1-azabicylco[2.2.2]octan-3-amine; adrenaline; amiodarone hydrochloride; atropine sulphate; diazepam; ephedrine; frusemide; haloperidol; lignocaine; metoclopramide; noradrenaline; omeprazole; ondansetron; phenytoin; vercuronium; acyclovir; amoxicillin; cefotetan; cefotaxime; metronidazole; cefuroxime; flucloxacillin; bupivacaine; cilazapril; amlodipine; felodipine; fesoterodine; isradipine; nifedipine; nimodipine; nisoldipine; cyclosporine; saquinavir; itraconazole; and ketoconazole.

In specific embodiments, the oxygen sensitive API includes one or more of dronabinol ((−)-trans-Δ⁹-tetrahydrocannabinol); epinephrine (also known as adrenalin or adrenaline); dopamine (3,4-dihydroxyphenethylamine); chlorpromazine (CPZ); captopril; azithromycin; pseudoephedrine; tiagabine; acitretin; rescinnamine; lovastatin; tretinoin; isotretinoin; simvastatin; ivermectin; verapamil; oxybutynin; hydroxyurea; selegiline; esterified estrogens; tranylcypromine; carbamazepine; ticlopidine; methyldopahydro; chlorothiazide; methyldopamine; naproxen; acetaminophen; erythromycin; bupropion; rifapentine; penicillamine; mexiletine; verapamil; diltiazem; ibuprofen; cyclosporine; saquinavir; morphine; sertraline; cetirizine; N-[[2-methoxy-5-(1-methyl)phenyl]methyl]-2-(diphenylmethyl)-1-azabicylco[2.2.2]octan-3-amine; adrenaline; amiodarone hydrochloride; atropine sulphate; diazepam; ephedrine; frusemide; haloperidol; lignocaine; metoclopramide; noradrenaline; omeprazole; ondansetron; phenytoin; vercuronium; acyclovir; amoxicillin; cefotetan; cefotaxime; metronidazole; cefuroxime; flucloxacillin; bupivacaine; methylphenidate; fesoterodine fumarate; morphine; hydromorphone; promethazine; dopamine; epinephrine; norepinephrine; esterified estrogen; ephedrine; pseudoephedrine; acetaminophen; ibuprofen; danofloxacin; erythromycin; penicillin; cyclosporine; methyldopate; cetirizine; diltiazem; verapamil; mexiletine; chlorothiazide; carbamazepine; selegiline; oxybutynin; vitamin A; vitamin B; vitamin C; L-cysteine; L-tryptophan; morphine; hydromorphone; promethazine; pseudoephedrine; tiagabine; acitretin; rescinnamine; lovastatin; tretinoin; isotretinoin; simvastatin; ivermectin; verapamil; oxybutynin; hydroxyurea; selegiline; esterified estrogens; tranylcypromine; carbamazepine; ticlopidine; methyldopahydro; chlorothiazide; methyldopa; naproxen; acetaminophen; erythromycin; bupropion; rifapentine; penicillamine; mexiletine; verapamil; diltiazem; ibuprofen; cyclosporine; saquinavir; morphine; sertraline; cetirizine; and N-[[2-methoxy-5-(1-methyl)phenyl]methyl]-2-(diphenylmethyl)-1-azabicylco[2.2.2]octan-3-amine.

In specific embodiments, the pH sensitive API includes one or more of desmopressin (DDAVP) (D-amino D-arginine vasopressin); vitamin D3 (cholecalciferol); omeprazole; and esomeprazole.

In specific embodiments, the light (UV) sensitive API includes one or more of penicillin (PCN); diazepam; tretinoin; isotretinoin; naproxen; erythromycin; diazepam; haloperidol; acyclovir; amlodipine; isradipine; nifedipine; promethazine; norepinephrine; promethazine; tretinoin; naproxen; digoxin; nitroglycerin; aminophylline; amphotericin B; chlorpheniramine maleate; chlorpromazine HCl; cisplatin; dacarbazine; diazoxide; diphenhydramine; dopamine hydrochloride; doxycycline hyclate; droperidol; epinephrine hydrochloride; fluorouracil; folic acid; furosemide; hydrocortisone; isoproterenol; levarterenol bitartrate; menadiol sodium diphosphate; methadone; morphine sulphate; naloxone; neostigmine methylsulfate; nitroprusside solution; phenylephrine hydrochloride; phytonadione; prochlorperazine edisylate; propranolol hydrochloride; streptomycin sulphate; sulfisoxazole diolamine; terbutaline; testosterone cypionate; triflupromazine hydrochloride; vinblastine; vincristine sulphate; vitamin B complex; dextroamphetamine; ciprofloxacin; clarithromycin; griseofulvin; itraconazole; ketoconazole; terbinafine; tetracycline hydrochloride; 1,4-dihydropyridines; 4-nerolidylcatechol; avobenzone; barnidipine; butyl methoxydibenzoylmethane; doxorubicin; fluoroquinolones; melatonin; naltrexone; cephalosporins; resveratrol; sericin; 3-hydroxyflavone; 4-methylbenzylidene camphor; 5-hydroxyflavones; antazoline; xylometazoline; nafazoline; ascorbic acid; carvedilol; cilnidipine; diclofenac; diflunisal; doxycycline; lansoprazole; manidipine; methotrexate; nicardipine; ofloxacin; oxolinic acid; phenylpropanoids; quercetin; ranitidine; rhein, sulfanilamide; and triprolidine.

In specific embodiments, the API having an unpleasant odor or taste includes one or more of aspirin (acetylsalicylic acid); penicillin (PCN); dipyridamole; procaine; diazepam; pseudoephedrine; oxybutynin; erythromycin; bupropion; ibuprofen; diazepam; ondansetron; acetaminophen; ibuprofen; alprazolam; chlorpheniramine maleate; diphenhydramine; ciprofloxacin; clarithromycin; diclofenac; ofloxacin; ranitidine; valerian extracts; isometheptene; bucillamine; azilsartan medoxomil, olmesartan medoxomil; benzalkonium chloride; diacetyl/2,3-butanedione; zinc acetate dihydrate; phenylpropanolamine hydrochloride; famotidine; pogostemi herba; guaifenesin; thymol; Eucalyptus oil; benzethonium chloride; theophylline; anticholesterolemic saponins; cimetidine; gabapentin; isoprothiolane; carbetapentane citrate; noscapine hydrochloride; quinine; L-leucine; iso-leucine; papaverine; talampicillin hydrochloride; indeloxazine hydrochloride; pinaverium bromide; propantheline bromide; triprolidine hydrochloride; dimenhydrinate; enoxacin; sparfloxacin; famotidine; amoxycillin trihydrate; morphine hydrochloride; amiprilose hydrochloride; terfenadine; beclamide; cetraxate; bifemelane hydrochloride; cefuroxime axetil; pirenzepine; nicorandil; levofloxacin; Gymnema sylvestre; buflomedil; orbifloxacin; chloroquine phosphate; famotidine; etoricoxib; pivoxil sulbactam; cetirizine dihydrochloride; cefpodoxime proxetil; desloratadine; dextromethorphan; amobarbital; sildenafil citrate; granisetron hydrochloride; levofloxacin; clopidogrel sulfate; telithromycin; pristinamycin; dihydrocodeine phosphate; potassium guaiacol sulfonate; fluoxetine; praziquantel; epsiprantel; risperidone; roxithromycin; bromhexine; paracetamol; pioglitazone; donepezil chloride; and sildenafil.

In specific embodiments, the water insoluble API includes one or more of dronabinol ((−)-trans-Δ⁹-tetrahydrocannabinol); diazepam; tretinoin; isotretinoin; carbamazepine; naproxen; ibuprofen; saquinavir, diazepam; nimodipine; acetaminophen; carbamazepine; carbamazepine; naproxen; ibuprofen; cyclosporine; saquinavir, estradiol; dexamethasone; dutasteride; doxercalciferol; calcitriol; tacrolimus; lorazepam; repaglinide; sirolimus; amphotericin B; griseofulvin; itraconazole; tetracycline hydrochloride; aprepitant; fenofibrate; paliperidone; aripiprazole lauroxil; progesterone; spironolactone; diosmin; celecoxib; halofantrine hydrochloride; ritonavir; meloxicam; nimesulide; danazol; glibenclamide; teniposide; propanidid; lopinavir; nabilone; etravirine; aprepitant; megestrol; nystatin; etomidate; flurbiprofen; propofol; clofazimine; paricalcitol; and tipranavir.

In specific embodiments, the API includes one or more of the APIs shown in the tables below.

ACTIVE PHARMACEUTICAL INGREDIENTS (APIs)

| | | | Release Mechanism | |
|---|---|---|---|---|
| ENCAPSULATED | | Low Bioavailability | (ER, CR, SR, MR, DR, | |
| POLYMERS | Unpleasant Taste | (BA) | PR, TR) | Unstable (O₂, UV, H₂O) |
| Hydroxypropyl | Acetaminophen | Vitamin B12 | Alprazolam | Psilocybin |
| methylcellulose (HPMC) | Aspirin | Iron | Metformin | Tetrahydrocannabinol |
| Ethylcellulose | Ibuprofen | Calcium | Metoprolol succinate | (THC) |
| Cellulose acetate phthalate | Chlorpheniramine | Magnesium | Tramadol | Clodinafop-propargyl |
| (CAP) | maleate | Zinc | Doxycycline hyclate | Acetamiprid |
| Eudragit L100 | Phenylpropanolamine | Provitamin A | Fluoxetine | Pinoxaden |
| Eudragit S100 | HCl | carotenoids | Omeprazole | Azimsulfuron |
| Eudragit FS30D | Famotidine | Vitamin K | Mesalamine | Bersulfuron |
| Shellac | Diclofenac | Folate | Risedronate | Chlorimuron |
| Polyvinyl acetate phthalate | Guaifenesin | Curcumin | Aspirin | Chlorsulfuron |
| (PVAP) | Ketoprofen | Many probiotics | NSAIDS | Cinosulfuron |
| Chitosan and its derivatives | H2-blockers | Fat-soluble vitamins (A, | Venlafaxine | Cyclosulfamuron |
| Pectin | Prednisolone | D, E, K) | Theophylline | Ethametsulfuron |
| Guar gum | Zinc acetate dihydrate | Magnesium oxide | Niacin | Ethoxysulfuron |
| Xanthan gum | Talampicillin HCl | Forms of iron | Diltiazem | Flazasulfuron |
| Polyvinylpyrrolidone (PVP) | Ciprofloxacin | supplements - | Oxycodone | Flupyrsulfuron |
| Polyethylene glycol (PEG) | Metronidazole | particularly iron oxide | Methylphenidate | Halosulfuron |
| Hydroxypropyl cellulose | Clarithromycin | Vitamin C (high doses) | Bupropion | Imazosulfuron |
| (HPC) | Amoxicillin | | Carbamazepine | Iodosulfuron |
| Polyvinyl alcohol (PVA) | Cephalexin | | Divalproex sodium | Mesosulfuron |
| Poly(acrylic acid) (PAA) | Erythromycin | | Lamotrigine | Metsulfuron |
| Poly(methacrylic acid-co- | Quinine | | Levetiracetam | Nicosulfuron |
| ethyl acrylate) | Caffeine | | Oxcarbazepine | Primisulfuron |
| Gelatin (used in combination | Pseudoephedrine | | Phenytoin sodium | Prosulfuron |
| with other polymers) | Dextromethorphan | | Topiramate | Pyrazosulfuron |
| Poly(lactic acid) (PLA) | Iron supplements (e.g., | | Furosemide | Rimsulfuron |
| Poly(lactic-co-glycolic acid) | ferrous sulfate) | | Propranolol | Sulfometuron |
| (PLGA) | Potassium chloride | | Isoniazide | Sulfosulfuron |
| Poly(ε-caprolactone) (PCL) | Vitamin B complex | | Erythromycin | Thifensulfuron |
| Alginate | Fish oil supplements | | Pancrelipase | Triasulfuron |
| Carbopol (polyacrylic acid) | Valerian root | | Pantoprazole | Tribenuron |
| Gellan gum | Garlic supplements | | Rabeprazole | Triflusulfuron |
| Poly(β-amino ester) | Probiotics | | Esomeprazole | Vitamin C (Ascorbic |
| Polyvinylacetal | β-Hydroxy β- | | Lansoprazole | acid) |
| diethylaminoacetate (AEA) | methylbutyric acid | | Budesonide | Vitamin A (Retinol) |
| Eudragit RS and RL | (HMB), otherwise | | Diclofenac sodium (in | Vitamin E |
| Poly(methyl methacrylate) | known as its conjugate | | combination with | (Tocopherols) |
| (PMMA) | base, β-hydroxy β- | | misoprostol) | Thiamine (Vitamin B1) |
| Hyaluronic acid | methylbutyrate | | Sulfasalazine - | Riboflavin (Vitamin B2) |
| Carboxymethyl cellulose | | | Azulfidine | Niacin (Vitamin B3) |
| (CMC) | | | Bisacodyl - Alophen | Pyridoxine (Vitamin |
| Polyurethanes | | | Mycophenolic acid | B6) |
| | | | Fish oil and omega-3 | Folic Acid (Vitamin B9) |
| | | | fatty acid supplements | Cobalamin (Vitamin |
| | | | Garlic supplements | B12) |
| | | | SAMe (S-Adenosyl | Probiotics |
| | | | methionine) | Omega-3 fatty acids |
| | | | Probiotics | Iron (in certain forms) |
| | | | Tegaserod maleate | Carotenoids |
| | | | Vitamin B1 | Polyphenols |
| | | | Metformin | |
| | | | Pregabalin | |
| | | | Phenylephrine | |
| | | | Bupropion | |
| | | | hydrochloride | |
| | | | Curcumin | |
| | | | Lactobacillus | |
| | | | rhamnosus NRRL 442 | |
| | | | Bifidobacterium | |
| | | | adolescentis 15703T | |
| | | | Akkermansia | |
| | | | muciniphila | |
| | | | Nizatidine | |
| | | | Ibuprofen | |
| | | | Dipyridamole | |
| | | | Pseudoephedrine | |
| | | | Urapidil | |
| | | | Bupropion | |
| | | | Carbamazepine | |
| | | | Divalproex sodium | |
| | | | Vitamin A (retinol) | |
| | | | Vitamin C | |
| | | | Vitamin D | |
| | | | Vitamin E (α- | |
| | | | tocopherol) | |
| | | | B-complex vitamins | |
| | | | (including B1, B2, B3, | |
| | | | B5, B6, B12, folate) | |
| | | | Calcium | |
| | | | Iron | |
| | | | Magnesium | |
| | | | Zinc | |
| | | | Selenium | |
| | | | Copper | |
| | | | Manganese | |
| | | | Chromium | |
| | | | Iodine | |
| | | | Coenzyme Q10 | |
| | | | Alpha lipoic acid | |
| | | | Lutein | |
| | | | Zeaxanthin | |
| | | | Lycopene | |
| | | | Lactobacillus species | |
| | | | Bifidobacterium species | |
| | | | Streptococcus species | |
| | | | Acetaminophen | |
| | | | Caffeine | |
| | | | Isosorbide mononitrate | |
| | | | Vancomycin | |
| | | | Pediococcus | |
| | | | pentosaceus Li05 | |
| | | | Yeast probiotics | |

Polymers suitable to encapsulate active pharmaceutical ingredients
(APIs) for various controlled release formulations

| | |
|---|---|
| 1 | Gelatin - Useful in combination with other polymers for pulsatile release systems. |
| 2 | Poly(lactic acid) (PLA) - Useful for controlled and pulsatile release formulations. |
| 3 | Poly(lactic-co-glycolic acid) (PLGA) - Useful for pulsatile and sustained release formulations. |
| 4 | Poly(ε-caprolactone) (PCL) - Useful for sustained release formulations. |
| 5 | Alginate - Useful for pH-dependent and sustained release formulations. |
| 6 | Carbopol (polyacrylic acid) - Useful for pH-sensitive and mucoadhesive drug delivery systems. |
| 7 | Gellan gum - Useful for controlled release and floating drug delivery systems. |
| 8 | Poly(β-amino ester) - Useful for pH-responsive intracellular delivery. |
| 9 | Polyvinylacetal diethylaminoacetate (AEA) - Useful for pH-dependent drug release. |
| 10 | Eudragit RS and RL - Useful for time-controlled release formulations. |
| 11 | Poly(methyl methacrylate) (PMMA) - Useful in combination with other polymers for controlled release. |
| 12 | Hyaluronic acid - Useful for targeted and controlled release formulations. |
| 13 | Carboxymethyl cellulose (CMC) - Useful in combination with other polymers for controlled release. |
| 14 | Polyurethanes - Useful for pH-responsive and controlled release formulations. |

APIs to be formulated with enteric coating

| | |
|---|---|
| 1 | Erythromycin - An antibiotic formulated as delayed-release capsules with enteric-coated pellets. |
| 2 | Pancrelipase - An enzyme used for digestion, formulated as delayed-release capsules |
| | containing enteric-coated spheres. |
| 3 | Proton pump inhibitors (PPIs): |
| | Pantoprazole (Protonix) |
| | Rabeprazole (Aciphex) |
| | Omeprazole (Prilosec) |
| | Esomeprazole (Nexium) |
| | Lansoprazole (Prevacid) |
| 4 | Budesonide (Entocort EC) - Used to treat Crohn's disease, formulated as capsules |
| | filled with enteric-coated granules. |
| 5 | Aspirin - Enteric-coated versions like Ecotrin are available to reduce stomach irritation. |
| 6 | Diclofenac sodium (in combination with misoprostol) - Arthrotec is an enteric-coated formulation. |
| 7 | Sulfasalazine - Azulfidine EN-tabs are enteric-coated. |
| 8 | Bisacodyl - Alophen is an enteric-coated version. |
| 9 | Mycophenolic acid (Myfortic) - An enteric-coated version of mycophenolate mofetil. |
| 10 | Fish oil and omega-3 fatty acid supplements - Some are enteric-coated to prevent fishy reflux. |
| 11 | Garlic supplements - Sometimes enteric-coated to prevent garlic breath and improve absorption. |
| 12 | SAMe (S-Adenosyl methionine) - Often enteric-coated due to its instability in acidic environments. |
| 13 | Some probiotic formulations - To protect the bacteria from stomach acid. |

APIs with low oral bioavailability (BA)

| | |
|---|---|
| 1 | Vitamin B12 - Absorption can be limited, especially in older adults or those with |
| | certain gastrointestinal conditions. |
| 2 | Iron - Particularly from plant sources, has low bioavailability (around 5-12% from a mixed diet). |
| 3 | Calcium - Only about 30% of calcium from food sources is absorbed. |
| 4 | Magnesium - Absorption rates can vary widely, but are generally low. |
| 5 | Zinc - Bioavailability can be low, especially from plant-based sources. |
| 6 | Provitamin A carotenoids (like β-carotene) - Only about 15.6% bioavailable from plant sources. |
| 7 | Vitamin K - Only about 16.5% bioavailable from plant sources. |
| 8 | Folate - From food sources, bioavailability can be limited. |
| 9 | Curcumin - Known for its poor bioavailability when taken orally. |
| 10 | Phylloquinone (Vitamin K1) - Less than 5% bioavailable from dark green leafy vegetables. |
| 11 | Many probiotics - Can have low survival rates through the acidic environment of the stomach. |
| 12 | Fat-soluble vitamins (A, D, E, K) - May have lower bioavailability when not |
| | consumed with dietary fats. |
| 13 | Magnesium oxide - A common form of magnesium supplement with poor absorption. |
| 14 | Some forms of iron supplements - Particularly iron oxide, which is poorly absorbed. |
| 15 | Vitamin C - While generally well-absorbed, high doses can lead to decreased bioavailability. |

APIs that degrade in the presence of oxygen

| | |
|---|---|
| 1 | Vitamin C (Ascorbic acid) - Highly susceptible to oxidation, especially in solution. |
| 2 | Vitamin A (Retinol) - Degrades through oxidation when exposed to oxygen. |
| 3 | Vitamin E (Tocopherols) - Sensitive to oxidation, especially in the |
| | presence of light and oxygen. |
| 4 | Thiamine (Vitamin B1) - Can degrade when exposed to oxygen, especially in liquid formulations. |
| 5 | Riboflavin (Vitamin B2) - Sensitive to oxidation, particularly when exposed to light. |
| 6 | Niacin (Vitamin B3) - Can degrade in the presence of oxygen, though less |
| | sensitive than some other vitamins. |
| 7 | Pyridoxine (Vitamin B6) - Susceptible to oxidation, especially in liquid forms. |
| 8 | Folic Acid (Vitamin B9) - Can degrade when exposed to oxygen and light. |
| 9 | Cobalamin (Vitamin B12) - Sensitive to oxidation, especially in liquid formulations. |
| 10 | Probiotics - Many probiotic strains are anaerobic or microaerophilic, |
| | meaning oxygen can be toxic to them. |
| 11 | Omega-3 fatty acids - Highly susceptible to oxidation, leading to rancidity. |
| 12 | Iron (in certain forms) - Can oxidize, potentially affecting bioavailability. |
| 13 | Carotenoids (like β-carotene) - Susceptible to oxidation, especially when exposed to light and oxygen. |
| 14 | Polyphenols - Many polyphenolic compounds can oxidize when exposed to air. |

pH Release Polymers

| | |
|---|---|
| 1 | Eudragit: A group of methacrylic acid copolymers used in oral drug delivery systems. Different types of Eudragit dissolve at |
| | different pH levels: |
| 2 | Eudragit L: Dissolves at pH above 6.0, typically used for enteric coatings to release drugs in the intestine. |
| 3 | Eudragit S: Dissolves at pH above 7.0, for targeting drug release in the colon. |
| 4 | Poly(acrylic acid) (PAA): This polymer is pH-sensitive due to its carboxylic acid groups, which ionize at higher pH, leading to |
| | swelling and drug release. |
| 5 | Poly(methacrylic acid-co-ethyl acrylate): Often used in enteric coatings to protect drugs from the acidic environment of the |
| | stomach and release them in the more neutral or basic environment of the intestine. |
| 6 | Chitosan: A natural polymer that is soluble in acidic conditions but becomes insoluble at neutral to alkaline pH, making it |
| | useful for pH-responsive drug delivery. |
| 7 | Poly(ethylene glycol) (PEG) conjugates: PEG can be attached to pH-sensitive polymers to create copolymers that release drugs |
| | in response to pH changes. |
| 8 | Poly(vinyl alcohol) (PVA) blends: PVA can be combined with other pH-sensitive polymers to create systems that release drugs |
| | in response to specific pH levels. |

Polymers for targeted delivery to the lower GI tract

| | |
|---|---|
| 1 | Cellulose derivatives: |
| | Hydroxypropyl methylcellulose (HPMC) |
| | Ethylcellulose |
| | Cellulose acetate phthalate (CAP) |
| 2 | Methacrylic acid copolymers: |
| | Eudragit L100 |
| | Eudragit S100 |
| | Eudragit FS30D |
| 3 | Other pH-sensitive polymers: |
| | Shellac |
| | Polyvinyl acetate phthalate (PVAP) |
| 4 | Biodegradable polymers: |
| | Chitosan and its derivatives |
| | Pectin |
| | Guar gum |
| | Xanthan gum |
| 5 | Synthetic polymers: |
| | Polyvinylpyrrolidone (PVP) |
| | Polyethylene glycol (PEG) |
| 6 | Time-dependent polymers: |
| | Hydroxypropyl cellulose (HPC) |
| | Polyvinyl alcohol (PVA) |

These polymers are often used in various combinations and formulations to achieve targeted delivery to the lower GI tract. They
work by resisting degradation in the upper GI tract (stomach and small intestine) due to their pH-sensitivity, enzymatic degradation
properties, or time-dependent release mechanisms. When the formulation reaches the lower GI tract, changes in pH, presence of
specific enzymes, or elapsed time trigger the release of the active ingredient.
APIs known to be water sensitive, that degrade in the presence of water

| | |
|---|---|
| 1 | Clodinafop-propargyl |
| 2 | Acetamiprid |
| 3 | Pinoxaden |
| 4 | Azimsulfuron |
| 5 | Bersulfuron |
| 6 | Chlorimuron |
| 7 | Chlorsulfuron |
| 8 | Cinosulfuron |
| 9 | Cyclosulfamuron |
| 10 | Ethametsulfuron |
| 11 | Ethoxysulfuron |
| 12 | Flazasulfuron |
| 13 | Flupyrsulfuron |
| 14 | Halosulfuron |
| 15 | Imazosulfuron |
| 16 | Iodosulfuron |
| 17 | Mesosulfuron |
| 18 | Metsulfuron |
| 19 | Nicosulfuron |
| 20 | Primisulfuron |
| 21 | Prosulfuron |
| 22 | Pyrazosulfuron |
| 23 | Rimsulfuron |
| 24 | Sulfometuron |
| 25 | Sulfosulfuron |
| 26 | Thifensulfuron |
| 27 | Triasulfuron |
| 28 | Tribenuron |
| 29 | Triflusulfuron |

Functional groups present on APIs that are prone to hydrolysis

| | |
|---|---|
| 1 | Esters: Carboxylic esters are highly susceptible to hydrolysis, especially in acidic or basic |
| | conditions in the body. Many prodrugs utilize ester linkages designed to be hydrolyzed to release the active drug. |
| 2 | Amides: While more stable than esters, amide bonds can still undergo hydrolysis, particularly in acidic environments like the |
| | stomach. For example, β-lactam antibiotics containing strained cyclic amide rings (lactams) are prone to hydrolysis. |
| 3 | Acetals and Ketals: These functional groups are susceptible to hydrolysis, especially |
| | in acidic conditions. Digoxin, for example, contains acetal groups prone to degradation. |
| 4 | Hemiacetals and Hemiketals: These are generally unstable and can readily convert to aldehydes or ketones in aqueous |
| | environments. |
| 5 | Imines (C═N): Found in drugs like diazepam, imines are susceptible to hydrolysis. |
| 6 | Alkyl halides: These can undergo nucleophilic substitution reactions with water or other nucleophiles in the body. |
| 7 | Phosphate esters: Found in drugs like hydrocortisone sodium phosphate, these can be |
| | hydrolyzed by phosphatase enzymes in the body. |
| 8 | Sulfate esters: Similar to phosphate esters, these can be cleaved by sulfatase enzymes. |
| | Heparin, for example, contains sulfate ester groups. |
| 9 | Anhydrides: Anhydrides are highly susceptible to hydrolysis. |
| 10 | Lactones: Cyclic esters that can undergo ring-opening hydrolysis. |
| 11 | Lactams - Cyclic amides, such as those found in β-lactam antibiotics like penicillins |
| | and cephalosporins, are particularly susceptible to hydrolysis. |
| 12 | Carbamates - These can hydrolyze to form carbamic acid, which then decomposes to carbon dioxide and an amine. |
| 13 | Imides - Cyclic compounds containing two carbonyl groups bound to nitrogen, which can undergo hydrolysis. |
| 14 | Nitriles - Can hydrolyze to form carboxylic acids, especially under acidic or basic conditions. |
| 15 | Epoxides - Three-membered cyclic ethers that can undergo ring-opening hydrolysis. |
| 16 | Ortho esters - Highly susceptible to acid-catalyzed hydrolysis. |
| 17 | Acid chlorides - Rapidly hydrolyze to form carboxylic acids. |
| 18 | Isocyanates - React with water to form carbamic acids, which then decompose to amines and carbon dioxide. |
| 19 | Thioesters - Similar to oxygen-based esters, but with sulfur instead of oxygen. |
| 20 | Azo compounds - Can undergo hydrolytic cleavage, especially in acidic conditions. |

APIs that have an unpleasant flavor

| | |
|---|---|
| 1 | Acetaminophen (paracetamol) - bitter taste |
| 2 | Aspirin - bitter and acidic taste |
| 3 | Ibuprofen - bitter taste |
| 4 | Chlorpheniramine maleate - bitter taste |
| 5 | Phenylpropanolamine HCl - bitter taste |
| 6 | Famotidine - bitter taste |
| 7 | Diclofenac - bitter taste |
| 8 | Guaifenesin - bitter taste |
| 9 | Ketoprofen - bitter taste |
| 10 | H2-blockers (e.g., ranitidine) - bitter taste |
| 11 | Prednisolone - bitter taste |
| 12 | Zinc acetate dihydrate - metallic taste |
| 13 | Talampicillin HC1 - unpleasant taste |
| 14 | Ciprofloxacin - bitter taste |
| 15 | Metronidazole - metallic taste |
| 16 | Clarithromycin - bitter taste |
| 17 | Amoxicillin - bitter taste |
| 18 | Cephalexin - bitter taste |
| 19 | Erythromycin - bitter taste |
| 20 | Quinine - extremely bitter taste |
| 21 | Caffeine - bitter taste |
| 22 | Pseudoephedrine - bitter taste |
| 23 | Dextromethorphan - bitter taste |
| 24 | Iron supplements (e.g., ferrous sulfate) - metallic taste |
| 25 | Potassium chloride - salty and bitter taste |
| 26 | Vitamin B complex - unpleasant taste |
| 27 | Fish oil supplements - fishy taste and odor |
| 28 | Valerian root - unpleasant odor and taste |
| 29 | Garlic supplements - strong odor and taste |
| 30 | Certain probiotics - can have a sour or unpleasant taste |

APIs suitable for pulsatile release, targeted release, sustained release, controlled release,
prolonged release, pH dependent release, enzyme triggered release, extended-release,
delayed-release, or modified-release

| | |
|---|---|
| 1 | Alprazolam - Extended release |
| 2 | Metformin - Extended release |
| 3 | Metoprolol succinate - Extended release |
| 4 | Tramadol - Extended release |
| 5 | Doxycycline hyclate - Delayed release |
| 6 | Fluoxetine - Delayed release |
| 7 | Omeprazole - Delayed release |
| 8 | Mesalamine - Delayed release |
| 9 | Risedronate - Delayed release |
| 10 | Aspirin - Delayed release |
| 11 | NSAIDs - Delayed release |
| 12 | Venlafaxine - Extended release |
| 13 | Theophylline - Extended release |
| 14 | Niacin - Extended release |
| 15 | Diltiazem - Extended release |
| 16 | Oxycodone - Extended release |
| 17 | Methylphenidate - Extended release |
| 18 | Bupropion - Extended release |
| 19 | Carbamazepine - Extended release |
| 20 | Divalproex sodium - Extended release |
| 21 | Lamotrigine - Extended release |
| 22 | Levetiracetam - Extended release |
| 23 | Oxcarbazepine - Extended release |
| 24 | Phenytoin sodium - Extended release |
| 25 | Topiramate - Extended release |
| 26 | Furosemide - Sustained release |
| 27 | Propranolol - Sustained release |
| 28 | Isoniazide - Controlled release |
| 29 | Diltiazem - Controlled release |

This list includes pharmaceuticals from various therapeutic categories, including antiepileptics, antidepressants, anxiolytics,
antidiabetics, pain medications, and others. The specific release mechanisms (e.g., pulsatile, targeted, pH-dependent, enzyme-
triggered) are not always explicitly stated in product names or common usage, but many of these formulations employ such
mechanisms to achieve their desired release profiles.
APIs suitable to be administered via pH dependent release

| | |
|---|---|
| 1 | Tegaserod maleate (TM) - Used for irritable bowel syndrome, formulated with pH-dependent |
| | polymers like Eudragit L100 and Eudragit S100 |
| 2 | Vitamin B1 (Thiamine) - Encapsulated for pH-dependent controlled release |
| 3 | Basic drugs - Generally exhibit pH-dependent solubility and dissolution profiles |
| 4 | Metformin - pH-dependent sustained release formulations |
| 5 | Beta-adrenoreceptor antagonists - pH-dependent sustained release formulations |
| 6 | Tramadol - pH-dependent sustained release formulations |
| 7 | Pregabalin - pH-dependent sustained release formulations |
| 8 | Phenylephrine - pH-dependent sustained release formulations |
| 9 | Bupropion hydrochloride - pH-dependent modified release formulations |

APIs suitable for enzyme triggered release

| | |
|---|---|
| 1 | Probiotics - Specifically, Escherichia coli MGl655 (EM) delivered using an enzyme-triggered release system |
| 2 | Curcumin - Formulated as a curcumin-cyclodextrin (CD-Cur) inclusion complex for |
| | enzyme-triggered controlled release to target the colon |
| 3 | β-Cyclodextrin (β-CD) - Used as a carrier for curcumin in an enzyme-triggered release system |
| 4 | Lactobacillus rhamnosus - probiotic encapsulation, which involves enzyme-triggered release mechanisms |

APIs suitable for release in the colon

| | |
|---|---|
| 1 | Mesalamine (5-aminosalicylic acid) |
| 2 | Budesonide |
| 3 | Sulfasalazine: A prodrug that releases mesalamine in the colon |
| 4 | Olsalazine: A prodrug that releases mesalamine in the colon |
| 5 | Balsalazide: A prodrug that releases mesalamine in the colon |
| 6 | Fruquintinib: metastatic colorectal cancer, though not specifically designed for colonic release |
| 7 | Etrasimod: ulcerative colitis, though not specifically designed for colonic release |
| 8 | Risankizumab-rzaa: for ulcerative colitis, though not specifically designed for colonic release |

APIs suitable for release in the lower GI tract

| | |
|---|---|
| 1 | Mesalamine (5-aminosalicylic acid) |
| 2 | Budesonide MMX: A corticosteroid formulated for release throughout the colon |
| 3 | Sulfasalazine: A prodrug that releases mesalamine in the colon |
| 4 | Olsalazine: A prodrug that releases mesalamine in the colon |
| 5 | Balsalazide: A prodrug that releases mesalamine in the colon |
| 6 | Linaclotide: Acts locally in the intestine to treat irritable bowel syndrome with |
| | constipation and chronic idiopathic constipation |
| 7 | Plecanatide: Acts locally in the GI tract to treat chronic idiopathic constipation |
| 8 | Rifaximin: An antibiotic that acts primarily in the gut and is used for traveler's |
| | diarrhea and irritable bowel syndrome with diarrhea |
| 9 | Vancomycin: Used to treat Clostridioides difficile infection in the colon |
| 10 | Fidaxomicin: An antibiotic that acts locally in the colon to treat C. difficile infections |
| 11 | Vedolizumab: While administered intravenously, it specifically targets the gut and is effective in the colon |
| 12 | Etrasimod: Used to treat ulcerative colitis, though not specifically designed for colonic release |
| 13 | Risankizumab-rzaa: Used to treat ulcerative colitis, though not specifically designed for colonic release |

While these APIs are taken orally, their formulations are specifically designed to target the lower GI tract, particularly the colon.
The release mechanisms can vary, including pH-dependent coatings, time-release formulations, and prodrug designs that rely on
colonic bacteria for activation

### Manufacturing Method

In specific embodiments, the semi-solid oral dosage form is manufactured using electrospinning techniques.

In specific embodiments, a needle-free, high-voltage electrospinning process is used to create a nanofibrous matrix incorporating the encapsulated API.

In specific embodiments, the oral thin film is comprised of nanofibers formed by needle-free electrospinning with encapsulated peptide APIs.

In specific embodiments, the dosage form is produced using micro-depositing, with APIs precisely placed within or atop the oral thin film.

In specific embodiments, stratified or layered regions of API are created within an oral thin film matrix by micro-depositing.

In specific embodiments, the oral thin film contains a mucoadhesive region formed by micro-deposition for accelerated buccal absorption.

In specific embodiments, the matrix is molded inside a secondary oral capsule for additional API protection and flexible dosing.

In specific embodiments, a gelatin or plant-derived capsule shell encloses the semi-solid oral thin film or gummy for secondary barrier function.

In specific embodiments, the thin film is contained within a color-coded, plant-derived capsule for batch authenticity verification.

In specific embodiments, a 3D printing process is employed for manufacturing to precisely control API and excipient distribution.

In specific embodiments, 3D printing is used to produce oral thin films with customizable flavor, dose, or release profiles.

In specific embodiments, a subdivision feature such as scoring or perforation is incorporated by 3D printing for precise dose splitting.

In specific embodiments, an automated, high-throughput, on-demand production line is used for semi-solid oral dosage forms.

In specific embodiments, microfluidic encapsulation provides continuous manufacturing to ensure batch-to-batch uniformity.

In specific embodiments, a twin-screw extrusion line applies the encapsulating polymer in situ during matrix mixing.

In specific embodiments, encapsulation integrity is verified in-line with real-time Raman, near-infrared, or fluorescent spectroscopy.

In specific embodiments, real-time image analysis verifies coating thickness during encapsulation by laser diffraction.

In specific embodiments, continuous quality monitoring is implemented using in-line spectroscopy at multiple manufacturing stages.

In specific embodiments, the encapsulation process or matrix casting is performed under inert (nitrogen or argon) atmosphere to protect oxygen- or moisture-sensitive APIs.

In specific embodiments, encapsulation of the API is performed under reduced humidity or dehumidified air to prevent water ingress.

In specific embodiments, the encapsulated API is protected from light during manufacture by processing in a dark or UV-blocked area.

In specific embodiments, the method of manufacture includes a lyophilization (freeze-drying) step after encapsulation for enhanced API stability.

In specific embodiments, drying of the matrix is performed using bottom-side substrate drying to prevent surface defects or inhomogeneity.

In specific embodiments, post-encapsulation crosslinking is achieved via UV curing, heat, or controlled humidity.

Formulation & Matrix Features

In specific embodiments, the matrix is engineered to a precise chewiness, hardness, or resilience for consistent organoleptic properties.

In specific embodiments, the gummy or film matrix's texture profiles such as springiness and adhesive force are specified within a defined range.

In specific embodiments, the oral thin film or gummy is measured to a specific dissolution pH for improved dissolution in the targeted region of the GI tract.

In specific embodiments, the matrix is manufactured to contain less than 1 wt % added sugar for health-conscious applications.

In specific embodiments, the dosage form is free of all major allergenic ingredients and comprises only GRAS or hypoallergenic excipients.

In specific embodiments, no animal-derived materials are used and the form is certified vegan and kosher.

In specific embodiments, the semi-solid dosage form is compounded with essential oils or natural antimicrobial agents for added oral care or shelf-life benefits.

In specific embodiments, the matrix incorporates prebiotics and probiotics together as a synbiotic combination.

In specific embodiments, the matrix includes plant-based colorants and flavors with no synthetic dyes.

In specific embodiments, the oral dosage form is a multi-compartment device, with separate matrices or gels in a single capsule or shell.

In specific embodiments, the multi-compartment dosage form distinctly separates different APIs for staged or synergistic release.

In specific embodiments, the matrix contains embedded API-coated beadlets or mini-tablets for combination therapy.

In specific embodiments, the oral thin film achieves rapid dissolution within 10-30 seconds for emergency or pediatric uses.

In specific embodiments, the matrix is manufactured to deliver a full therapeutic dose in under 1 minute.

In specific embodiments, films or gummies provide a biphasic release with both an immediate and a sustained API plasma profile.

API & Loading Features

In specific embodiments, ultra-low doses (sub-microgram) of highly potent APIs are enabled by encapsulation and uniform mixing.

In specific embodiments, ultra-high doses of mild APIs (>1 g per unit) are enabled by encapsulation to avoid taste or texture issues.

In specific embodiments, the dosage form comprises a chemotherapeutic or cytotoxic API at a precisely controlled ultra-low dose.

In specific embodiments, the oral thin film or gummy contains up to five or more different actives, each with independent encapsulation and release.

In specific embodiments, fixed-dose combination products are included with both pharmaceutical and nutritional APIs.

In specific embodiments, the dosage form contains a vaccine antigen stabilized in a pH-sensitive encapsulation.

In specific embodiments, the encapsulation provides isolation for both the API and key flavor or aroma agents to mask unpleasant tastes.

In specific embodiments, a cyclodextrin-based micro-encapsulation is used for flavor or odor masking.

In specific embodiments, aroma-masking is further enhanced by multi-layer encapsulation with essential oil or adsorbent layers.

In specific embodiments, the encapsulation includes a co-encapsulated absorption enhancer such as bile salts, fatty acids, or surfactants optimized for difficult APIs.

In specific embodiments, lipid-based excipient blends within the encapsulation enhance solubility of validated poorly bioavailable APIs.

In specific embodiments, the encapsulated API is a biologic or gene therapy that is stabilized by a nucleic acid binding polymer, such as poly-L-lysine or chitosan.

In specific embodiments, the encapsulated API is protected by both a reducing agent (glutathione, dithiothreitol, vitamin C) and a UV-barrier additive.

In specific embodiments, the dosage form is formulated for enzyme-triggered release exclusively after exposure to colonic bacteria.

In specific embodiments, the encapsulation includes cell-penetrating peptides, lectins, or targeting ligands for specific GI tract uptake.

In specific embodiments, the matrix includes bioadhesive or mucoadhesive agents for extended mucosal residence and absorption.

Device, Traceability, & Compliance Features

In specific embodiments, a machine-readable, edible, or imperceptible QR code, RFID, or similar marker is present in each dosage unit for digital traceability.

In specific embodiments, each dosage unit's QR code is readable by a smartphone for medication management.

In specific embodiments, RFID markers in packaging or units provide automatic inventory and anti-counterfeiting capabilities.

In specific embodiments, unit dose blisters or bottles are compatible with automated digital medication dispensers.

In specific embodiments, the form factor is compatible with ingestible digital sensors for real-time ingestion verification.

In specific embodiments, packaging includes child-resistant and tamper-evident features.

In specific embodiments, the oral dosage form is compatible with medical device sensors for inventory, patient adherence, or automated reminders.

In specific embodiments, the matrix includes an optical or imaging tracer so unit passage through the GI tract can be monitored non-invasively by MRI, CT, or fluorescence.

In specific embodiments, the encapsulation incorporates a radio-opaque or iron-oxide label for radiographic transit studies.

### Special Populations/Regulatory/Therapeutic Features

In specific embodiments, the matrix is scored into subunits for dose titration or

pediatric/geriatric splitting.

In specific embodiments, subdividable units are perforated for easy splitting by hand without tools.

In specific embodiments, the dose per segment is precisely indicated for dose titration safety.

In specific embodiments, the matrix/excipients are sensorially optimized-flavors/textures validated in children and elderly.

In specific embodiments, the oral dosage form is palatable to veterinary patients (dogs, cats), with tailored animal flavors and safety profiles.

In specific embodiments, the formulation and packaging meet all major regulatory standards (FDA, EMA, ICH, cGMP, etc.) with documented batch-to-batch uniformity.

In specific embodiments, the packaging supports stability for >36 months at variable temperature and humidity extremes.

In specific embodiments, the form is suitable for direct use in clinical trials or decentralized clinical study designs.

In specific embodiments, the encapsulation is analyzed by both differential scanning calorimetry and X-ray diffraction.

In specific embodiments, encapsulation uniformity is verified using both electron microscopy and tracer molecule testing.

In specific embodiments, the matrix contains mini-tablets or layered films for platforms requiring more than three release phases.

In specific embodiments, the packaging is compostable, paper-based, and contains a humidity barrier layer of plant-based polylactide.

### Dedicated Taste- and Aroma-Masking Outer Layer Over Encapsulation

In specific embodiments, the dosage form comprises an encapsulation system that includes a dedicated outer taste- or aroma-masking layer.

In specific embodiments, the dosage form comprises an encapsulated API, wherein the encapsulation includes a taste-masking polymeric outer layer physically distinct from a core stabilizing layer.

In specific embodiments, the oral thin film or gummy comprises an API encapsulated by a dual-layer system, the outermost of which comprises cyclodextrin or zeolite for odor and taste masking.

### Stratified (Layered) Matrix for Segregated API Release Regions

In specific embodiments, the oral dosage form comprises a stratified polymer matrix having discrete spatial regions with varying release profiles.

In specific embodiments, the oral thin film or gummy comprises physically separate layers, one with immediate release and at least one with delayed or controlled release properties.

In specific embodiments, the oral thin film is constructed as a three-layer assembly, with at least one intermediate layer containing enterically-encapsulated API and surface layers for immediate release.

### API Micro-Deposition (Spot or Pattern Loading) Technology

In specific embodiments, the oral dosage form comprises at least one API deposited by micro-depositing technology.

In specific embodiments, the oral thin film incorporates APIs localized in discrete zones by micro-deposition, separate from the general matrix.

In specific embodiments, the oral thin film contains precise micro-deposited spots of API on or within one film surface, providing for localized buccal or sublingual absorption.

Encapsulation System with Mucoadhesive or Targeting Moieties

In specific embodiments, the encapsulation comprises a polymer functionalized with moieties for tissue targeting or mucoadhesion.

In specific embodiments, the encapsulation polymer is chemically conjugated with a mucoadhesive group, such as lectin or folic acid.

In specific embodiments, the encapsulation system comprises a biopolymer shell containing conjugated bioadhesive ligands that promote targeted retention on oral or intestinal mucosal surfaces.

Encapsulation/Matrix Combination with Nanoscale Additives (e.g., Nanoparticles/Nanoclusters)

In specific embodiments, the encapsulation and/or matrix incorporates nanoscale additives.

In specific embodiments, the encapsulation shell includes nanoparticles of titanium dioxide, iron oxide, or silica for enhanced stability or imaging.

In specific embodiments, the encapsulation comprises a hybrid layer incorporating polymer and nanoparticles (mean size <50 nm) to create controlled permeability for moisture and oxygen.

### Enzyme-Triggered Release Exclusively Dependent on Colonic Bacteria

In specific embodiments, the encapsulation is formulated to release API exclusively in response to the enzymatic activity of colonic bacterial flora.

In specific embodiments, a polysaccharide-based encapsulation shell is fabricated to degrade only upon exposure to colonic glycosidases.

In specific embodiments, the dosage form is designed such that the encapsulation remains intact through the stomach and small intestine and undergoes degradation exclusively via β-galactosidase from colonic microbiota.

### Self-Emulsifying or Self-Dispersing Encapsulation for Low-Solubility APIs

In specific embodiments, the encapsulation comprises a self-emulsifying polymer system to increase solubility of the API upon GI release.

In specific embodiments, the encapsulation formulation comprises surfactant(s) and oil phase(s) that spontaneously form a microemulsion in GI fluid.

In specific embodiments, a poorly water-soluble API is encapsulated with a combination of poloxamer surfactant and medium-chain triglycerides, forming a self-emulsifying system on release.

API Encapsulation with Multi-Compartment (Multi-Layer or Multi-Core) Architecture

In specific embodiments, the API is encapsulated within a multi-compartment, multi-core, or multi-layer structure.

In specific embodiments, the encapsulation comprises two or more concentric polymer shells with different permeability or solubility characteristics.

In specific embodiments, the encapsulation comprises an inner core containing API and absorption enhancer, an intermediate time-dependent polymer layer, and an outer pH-sensitive release shell.

### In-Situ Post-Encapsulation Crosslinking

In specific embodiments, the encapsulation coating is crosslinked in situ after API coating or embedding.

In specific embodiments, the encapsulation polymer is crosslinked by UV, visible light, heat, or chemical initiator after matrix formation.

In specific embodiments, an encapsulated API within a gummy matrix is post-processed by photo-initiated crosslinking of the encapsulation shell to increase barrier stability.

### Diagnostic or Imaging Agent Embedded Within Encapsulation/Matrix

In specific embodiments, the dosage form comprises a diagnostic or imaging agent embedded in the matrix or encapsulation.

In specific embodiments, the encapsulation layer incorporates iron oxide or barium sulfate for radiographic or MRI imaging of GI transit.

In specific embodiments, the dosage form comprises an encapsulated nanoparticle tracer (mean diameter <100 nm, Fe3O4) in the polymer shell for quantification by medical imaging after oral administration.

### On-Demand Customization (Personalization)

In specific embodiments, the composition is personalized or customized on demand for the target user.

In specific embodiments, the ratio of encapsulated-to-unencapsulated API, flavor, or excipient content is varied based on patient need at the point of manufacture.

In specific embodiments, an automated manufacturing line enables adjustment of dose strength, flavor, and release profile based on user prescription data, with print-on-demand labeling.

### Flavors/Excipients/Matrix Materials Optimized for Non-Human (Veterinary) Palatability

In specific embodiments, the dosage form is designed for veterinary administration with species-specific palatability.

In specific embodiments, the matrix composition includes animal-attractant flavors and excipients suitable for canine or feline oral acceptance.

In specific embodiments, an antiparasitic agent is encapsulated and blended in a gummy containing hydrolyzed poultry flavor, free of artificial sweeteners or known animal allergens.

### Design for High-Temperature/High-Humidity Stability (Extreme Storage/Transport)

In specific embodiments, the dosage form is engineered for stability under extremes of temperature and humidity.

In specific embodiments, the encapsulation system remains stable to API loss at temperatures up to 60° C. and relative humidity up to 90%.

In specific embodiments, the encapsulation and packaging system maintains >90% API potency after continuous storage for four weeks at 55° C./90% RH.

Real-Time, in-Line Analytical Process Verification (Quality Control Integration)

In specific embodiments, the encapsulation and/or matrix is verified by in-line, real-time analytical process controls.

In specific embodiments, Raman, NIR, or hyperspectral imaging is used for continuous, automated monitoring of encapsulation integrity during manufacture.

In specific embodiments, every batch unit is scanned by in-line Raman spectroscopy to confirm polymer layer thickness and distribution uniformity prior to release.

### Unit-Dose Level Traceability (Machine-Readable Codes or Digital Markers Physically Embedded)

In specific embodiments, each dosage unit contains an embedded traceable marker for supply chain authentication.

In specific embodiments, the dosage matrix physically embeds a machine-readable batch or unit identifier, such as a microscopic QR code or color-coded bead.

In specific embodiments, the oral thin film matrix includes a visually imperceptible edible QR code readable with consumer devices to confirm authenticity and dosage history.

Encapsulation of Biologics with Preserved Activity (Protein, Peptide, RNA, Probiotics)

In specific embodiments, the encapsulation system is suitable for stabilizing biologics, such as proteins, peptides, RNA, or living cells.

In specific embodiments, a reducing agent and protease/RNase inhibitor is included within the encapsulation shell to protect the API until release in the target GI location.

In specific embodiments, the encapsulation preserves at least 80% measured bioactivity of a probiotic, enzyme, or mRNA API after exposure to gastric fluid for 1 hour.

### Design for Rapid-Dissolving, Emergency or Ultra-Fast Onset Use

In specific embodiments, the dosage form is designed for rapid dissolution and/or fast pharmacodynamic onset.

In specific embodiments, the oral thin film or gummy is engineered to completely disintegrate or dissolve within 30 seconds of oral administration.

In specific embodiments, a buccal thin film delivering an anxiolytic or antiepileptic API achieves greater than 90% dissolution and systemic absorption within one minute of oral contact.

Encapsulation of APIs with Hydrolysis-Prone Functional Groups Specifically Protected

In specific embodiments, APIs bearing functional groups prone to hydrolysis are protected by encapsulation.

In specific embodiments, APIs with at least one ester, amide, or lactam group are encapsulated by a polymer barrier that blocks water and acid ingress during manufacturing and storage.

In specific embodiments, a β-lactam antibiotic is encapsulated in a dual-layer shell of ethylcellulose and enteric methacrylic acid copolymer to block hydrolytic degradation until intestinal release.

Subdividable Dosage Unit with Precisely Marked Titration Features

In specific embodiments, the dosage form is physically subdividable for dose titration or personalization.

In specific embodiments, the matrix contains scored or perforated edges, with each segment containing a calibrated amount of API for flexible dosing.

In specific embodiments, the oral thin film is imprinted with division marks at 25%-increments, and each section is precisely quantified for API content within +5%.

### Packaging Solution Providing Both Barrier Properties and Environmental Compliance

In specific embodiments, the dosage form is packaged using a material conferring environmental sustainability and physical protection.

In specific embodiments, the packaging consists of a biodegradable, compostable, or recyclable material with embedded high-barrier properties to moisture and oxygen.

In specific embodiments, the dosage form is released in individual compartments of a paper-based blister pack lined with biodegradable PLA, providing <2% moisture ingress over 24 months.

### Enumerated Embodiments

Specific enumerated embodiments <1> to <54> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

<Embodiment 1> A semi-solid oral dosage form comprising one or more excipients, an active ingredient, and a polymer. The polymer encapsulates at least a portion of the active ingredient, and the portion of the active ingredient that is encapsulated does not come in direct contact with the one or more excipients present in the semi-solid oral dosage form. The semi-solid oral dosage form can be, e.g., an oral thin film (OTF) or gummy.

<Embodiment 2> A semi-solid oral dosage form comprising one or more excipients, an active ingredient, and a polymer, wherein the oral dosage form is selected from an oral thin film (OTF) and a gummy and the polymer encapsulates at least a portion of the active ingredient, the portion of the active ingredient that is encapsulated does not come in direct contact with the one or more excipients.

<Embodiment 3> A semi-solid oral dosage form, comprising an encapsulated active ingredient wherein the encapsulation fully surrounds the active ingredient, the encapsulation comprises at least one polymer selected from cellulose derivatives or methacrylic acid copolymers, and the dosage form is pharmaceutically or nutraceutically acceptable for human or animal ingestion in a format selected from gummies, oral thin films, pastilles, or chewable gels, with at least 2 wt. % water in the matrix.

<Embodiment 4> A semi-solid dosage form comprising a plurality of different active pharmaceutical ingredients, wherein each active ingredient is independently encapsulated using a distinct polymeric encapsulation providing separate release profiles, the matrix is gelatin-free, and the form is suitable for pediatric, geriatric, or dysphagic populations.

<Embodiment 5> A dosage form in which at least 90% of the total API content is encapsulated by a polymer matrix selected to be substantially impermeable to water, acid, and oxygen under conditions of manufacture, storage, and oral administration for up to 24 months at ambient temperature and humidity.

<Embodiment 6> A semi-solid oral dosage form comprising a core and a shell, wherein the core contains a gummy or oral thin film with an encapsulated active ingredient and the shell comprises a water-insoluble, flavor-masking film, such that the completed dosage unit resists water ingress during storage and releases the active ingredient only upon exposure to gastrointestinal enzymatic activity or pH changes above 6.5.

<Embodiment 7> A dosage form wherein at least one encapsulated active ingredient is a peptide, protein, or polynucleotide, and the encapsulation polymer includes a protease inhibitor or RNase inhibitor, such that at least 80% of biological activity is retained after exposure to gastric fluid for 1 hour.

<Embodiment 8> A semi-solid oral dosage form comprising an encapsulated prebiotic, probiotic, or combination thereof, wherein the encapsulation protects both live and non-live biologicals from acidic, oxidative, and hydrolytic degradation, and ensures at least 70% survival or potency following two months at 30° C. and 65% relative humidity.

<Embodiment 9> A dosage form comprising a semi-solid matrix, an encapsulated active ingredient, and a polymeric encapsulant comprising at least one mucoadhesive ligand, wherein the encapsulated active is released over a duration of at least four hours after oral administration as measured in simulated gastrointestinal conditions.

<Embodiment 10> An oral thin film dosage form, comprising a polymeric film matrix, at least one encapsulated hydrophobic active ingredient, and a surface-applied edible overcoat, wherein the overcoat is selected from fatty acid esters, shellac, or pectin and imparts both water-resistance and taste-masking properties to the film.

<Embodiment 11> A dosage form comprising a semi-solid oral thin film or gummy, wherein the encapsulation polymer is functionalized with at least one targeting ligand selected for specific binding to a site in the human gastrointestinal tract, and wherein release of the active ingredient is site-specific as demonstrated by in vitro dissolution testing at relevant pH and enzymatic conditions.

<Embodiment 12> A gummy or oral film dosage form wherein at least 50% of the active ingredient content is encapsulated by a pH-sensitive polymer combination selected such that a first population of encapsulated active dissolves at pH above 6.0 and a second population at pH above 7.0, thereby providing sequential site-specific release of the active at multiple intestinal locations.

<Embodiment 13> A semi-solid oral dosage form, wherein the encapsulation polymer comprises a blend of a biodegradable polyester and a time-dependent swelling polymer, providing programmed delayed release of the encapsulated API after a predetermined lag time of at least 30 minutes post-ingestion under simulated gastrointestinal conditions.

<Embodiment 14> A dosage form wherein at least 30% of the encapsulated active ingredient comprises a fat-soluble vitamin or caroteinoid, the encapsulation polymer includes a permeability enhancer, and the form further comprises an oil component in the matrix to increase oral bioavailability of the active as measured by pharmacokinetic analysis in a mammalian subject.

<Embodiment 15> A semi-solid oral dosage form comprising encapsulated particulates of a water-sensitive or acid-sensitive active ingredient, wherein encapsulation is achieved by a two-step process involving in situ crosslinking of an edible polymer, followed by overcoating with a pH-dependent release polymer, and where at least 70% of total API is encapsulated by both layers.

<Embodiment 16> A semi-solid oral dosage form comprising multiple encapsulated actives, wherein at least one encapsulated ingredient comprises a flavor or aroma-masking agent selected from cyclodextrins or encapsulated essential oils, and the matrix contains less than 1 wt. % added traditional sweeteners.

<Embodiment 17> An oral thin film or gummy dosage form, wherein API particles are encapsulated at a median particle size of less than 100 μm and dispersed in the matrix such that API content uniformity varies by less than 10% between individual dosage units as verified by analytical method.

<Embodiment 18> A semi-solid oral dosage form wherein the encapsulation comprises at least one enteric polymer layer and an inner layer of a reducing agent to prevent oxidative degradation of the API, and wherein the dosage form maintains at least 90% API potency after three months under accelerated stability conditions (40° C., 75% RH).

<Embodiment 19> A dosage form comprising an encapsulated active ingredient and an embedded digital marker, wherein the marker is detectable by consumer electronic devices and is contained within a visually imperceptible layer of the dosage unit, permitting subsequent identification or traceability during resale or use.

<Embodiment 20> A semi-solid oral dosage form, wherein the encapsulated API includes a prodrug form and the encapsulation is configured to release the prodrug in the colon via enzyme-triggered hydrolysis, with at least 60% of the released API bioactivated in simulated colonic fluid over two hours.

<Embodiment 21> A gum, gummy, or soft chew dosage form for veterinary administration, comprising at least one encapsulated active ingredient selected from antiparasitic agents, the encapsulation polymer tailored to resist canine or feline salivary hydrolysis and release the active in the animal's small intestine.

<Embodiment 22> A semi-solid dosage form wherein the encapsulated API is an mRNA, gene editing, or gene therapy agent formulated with an encapsulant comprising at least one biodegradable, cationic polymer and a nucleic acid stabilizer, such that mRNA integrity is preserved and measurable protein expression is achieved in a mammalian animal model after oral administration.

<Embodiment 23> A semi-solid oral dosage form comprising at least one encapsulated live probiotic organism, wherein the encapsulation process involves microfluidic droplet generation into a pH-sensitive gelling bath, yielding particles with mean diameter less than 20 μm, and the dosage form achieves at least 80% survival of viable cells after manufacture and two-month storage at room temperature.

<Embodiment 24> An oral thin film containing both encapsulated hydrophobic and water-soluble actives in distinct microcapsules, wherein each type is encapsulated using a different polymer chosen for targeted dissolution at two different sites in the gastrointestinal tract.

<Embodiment 25> An oral dosage form in which the encapsulating polymer is crosslinked via photoinitiated reaction using visible or UV light after the encapsulated API is embedded within the semi-solid matrix, such that the crosslinked shell enhances both barrier function and release control compared to non-crosslinked analogs.

<Embodiment 26> A semi-solid oral dosage form incorporating encapsulated biologics or RNA accompanied by peptide, antibody, or sugar-based ligands grafted onto the encapsulation shell, for the express purpose of targeting M cells or Peyer's patches in the intestinal epithelium, as demonstrated by increased bioavailability in an ex vivo gut tissue assay.

<Embodiment 27> A semi-solid dosage form, wherein the encapsulation process is performed continuously using a twin-screw extrusion system that disperses the API or APIs into a matrix while simultaneously applying a solvent-free, in situ forming polymethacrylate coating, and wherein mean coating thickness is between 15 μm and 150 μm.

<Embodiment 28> A gummy or oral thin film dosage form wherein the encapsulation polymer is blended in situ with a functionalized nanoparticle suspension, and wherein the resulting hybrid encapsulation achieves enhanced permeability barrier function and controlled, multi-phase API release that is not achievable by the polymer or nanoparticle alone.

<Embodiment 29> A solid or semi-solid oral dosage form comprising API encapsulation using a blend of synthetic polyacrylate and biodegradable polysaccharide derivatives, the resulting shell having both pH-dependent solubility and enzyme-triggered degradability, with not more than 5 wt. % residual organic solvents in the final product as verified by gas chromatography.

### Encapsulation Features

pH-Sensitive Polymer Encapsulation

<Embodiment 30> The dosage form or any one of <Embodiment 1> to <Embodiment 29> wherein the encapsulating polymer is selected from Eudragit L100, Eudragit S100, or cellulose acetate phthalate (CAP), and dissolves only at pH above 6.0 (intestine-targeted) or above 7.0 (colon-targeted).

### Multiple Discrete Polymer Layers

<Embodiment 31> The dosage form or any one of <Embodiment 1> to <Embodiment 30> wherein the encapsulation comprises at least two discrete polymer layers, each independently selected to protect against a different destabilizing agent (e.g., water, acid, oxygen, UV).

### Water-, Acid-, Oxygen-, and UV-Barrier Coating

<Embodiment 32> The dosage form or any one of <Embodiment 1> to <Embodiment 31> wherein the encapsulation demonstrates by tracer diffusion or permeability testing the substantial exclusion of water, acid, oxygen, and/or light over a defined storage interval (such as two weeks at accelerated conditions).

### Enzyme-Degradable or Bacteria-Sensitive Polymers

<Embodiment 33> The dosage form or any one of <Embodiment 1> to <Embodiment 32> wherein the encapsulation is configured to degrade or become permeable only in the presence of specific gastrointestinal enzymes or colonic bacteria, enabling targeted colonic or lower-GI delivery.

### Controlled Coating Thickness

<Embodiment 34> The dosage form or any one of <Embodiment 1> to <Embodiment 33> wherein the encapsulation layer(s) have an average thickness of between 10 μm and 300 μm, as measured by microscopy or image analysis.

### Mucoadhesive or Targeting Ligands

<Embodiment 35> The dosage form or any one of <Embodiment 1> to <Embodiment 34> wherein the encapsulation polymer is functionalized with mucoadhesive moieties or targeting ligands for site-specific retention or absorption in the GI tract (e.g., lectins, folate, or antibodies).

### Smart/Stimulus-Responsive Polymers

<Embodiment 36> The dosage form or any one of <Embodiment 1> to <Embodiment 35> wherein the encapsulation comprises polymers that are responsive to external or internal triggers such as temperature, light, magnetic field, ultrasound, or ionic strength.

### Blend of Natural and Synthetic Polymers

<Embodiment 37> The dosage form or any one of <Embodiment 1> to <Embodiment 36> wherein the encapsulation consists of a blend of at least one natural (e.g., polysaccharide) and one synthetic polymer (e.g., methacrylic acid copolymer).

### Dosage Form & Matrix Features

### Semi-Solid Dosage Forms: Gummies and Oral Thin Films Only

<Embodiment 38> The dosage form or any one of <Embodiment 1> to <Embodiment 37> wherein the oral dosage form is specifically a gummy or oral thin film (OTF), and the matrix comprises at least 4 wt. % water.

### Immediate and Modified Release Portions

<Embodiment 39> The dosage form or any one of <Embodiment 1> to <Embodiment 38> wherein the form includes both unencapsulated API (for immediate release) and encapsulated API (for delayed, extended, or targeted release).

### Overcoating or External Film Barrier

<Embodiment 40> The dosage form or any one of <Embodiment 1> to <Embodiment 39> wherein the finished dosage form is overcoated with an edible, water-resistant, oxygen-barrier, or taste-masking film.

### Multi-Compartment or Multi-API Design

<Embodiment 41> The dosage form or any one of <Embodiment 1> to <Embodiment 40> wherein the dosage form contains two or more APIs, each independently encapsulated or co-encapsulated, allowing for distinct or staged release profiles.

### Particle Size and Distribution of Encapsulated API

<Embodiment 42> The dosage form or any one of <Embodiment 1> to <Embodiment 41> wherein the mean particle size of the encapsulated API is between 1 μm and 300 μm, and content uniformity varies by less than 10% between units.

### Inclusion of Functional Excipients

<Embodiment 43> The dosage form or any one of <Embodiment 1> to <Embodiment 42> wherein the oral dosage form further comprises excipients selected from flavoring agents, sweeteners, colorants, acidulants, surfactants, fillers, plasticizers, and preservatives, each at FDA-permitted levels and suitable for pediatric, geriatric, or special-needs patients.

### Antioxidant or Reducing Agent in Encapsulation

<Embodiment 44> The dosage form or any one of <Embodiment 1> to <Embodiment 43> wherein the encapsulation includes a pharmaceutically acceptable antioxidant or reducing agent (e.g., ascorbic acid, glutathione) to protect against oxidative degradation of the API.

### Bioadhesive or Mucoadhesive Matrix Excipients

<Embodiment 45> The dosage form or any one of <Embodiment 1> to <Embodiment 44> wherein the matrix includes bioadhesive or mucoadhesive components (e.g., chitosan, carbomer, polycarbophil) to enhance absorption or retention.

### Product/Manufacturing & Analytical Controls

### Process Stability: High-Temperature Compatibility

<Embodiment 46> The dosage form or any one of <Embodiment 1> to <Embodiment 45> wherein the encapsulation and dosage form remain intact and API-stable after exposure to manufacturing temperatures up to 95° C.

### Automated In-Line Quality Control

<Embodiment 47> The dosage form or any one of <Embodiment 1> to <Embodiment 46> wherein the manufacturing process includes in-line or real-time monitoring (e.g., Raman spectroscopy, near-infrared imaging) to verify encapsulation integrity, coating thickness, or API uniformity.

### Analytical Verification of Isolation

<Embodiment 48> The dosage form or any one of <Embodiment 1> to <Embodiment 47> wherein the encapsulation integrity and isolation are verified by at least one analytical method including scanning electron microscopy, confocal microscopy, tracer diffusion, or permeability studies.

### Accelerated and Long-Term Stability

<Embodiment 49> The dosage form or any one of <Embodiment 1> to <Embodiment 48> wherein the dosage form retains at least 90% API potency when stored at 40° C. and 75% relative humidity for three months, or 25° C. and 60% RH for at least 24 months.

### Vegan, Allergen-Free, or Customizable Formulation

<Embodiment 50> The dosage form or any one of <Embodiment 1> to <Embodiment 49> wherein the matrix, encapsulation polymers, and excipients are selected to be free of major allergens, gelatin, and animal derivatives, and may use pectin, agar, or other plant-based alternatives.

### Traceability and Anti-Counterfeiting Technologies

<Embodiment 51> The dosage form or any one of <Embodiment 1> to <Embodiment 50> wherein each dosage unit or batch includes a machine-readable code, visual marker, digital adherence sensor, or edible QR code for supply chain authentication and traceability.

### Functional Marker or Imaging Agent

<Embodiment 52> The dosage form or any one of <Embodiment 1> to <Embodiment 51> wherein the dosage form includes an imaging tracer, dye, or diagnostic agent embedded for non-invasive confirmation of GI transit or compliance.

### Subdividable or Titrated Dosing Features

<Embodiment 53> The dosage form or any one of <Embodiment 1> to <Embodiment 52> wherein the dosage unit is provided with scoring or perforations allowing splitting for dose titration or personalization by patients or caregivers.

Compatibility with Pediatric, Geriatric, or Veterinary Use

<Embodiment 54> The dosage form or any one of <Embodiment 1> to <Embodiment 53> wherein the formulation and flavor, size, or texture are adapted for specific populations, and the excipient profile supports broad age and species acceptability.

1. A semi-solid oral dosage form comprising one or more excipients, an active ingredient, and a polymer,
wherein,
the oral dosage form is selected from an oral thin film (OTF) and a gummy;
the polymer encapsulates at least a portion of the active ingredient, such that at least 50% of the active ingredient is encapsulated,
the encapsulation forms a continuous polymeric barrier physically isolating at least the encapsulated portion of the active ingredient from the matrix excipients under storage and use condition;
the polymer encapsulating the active ingredient comprises a pH-sensitive polymer comprising at least one of Eudragit L100, Eudragit S100, and cellulose acetate phthalate (CAP), such that release of the active ingredient is targeted to the intestine or colon;
the encapsulation layer has an average thickness of between 10 μm and 300 μm as measured by microscopy or image analysis;
the portion of the active ingredient that is unencapsulated provides for immediate release, and the portion that is encapsulated provides for delayed, sustained, or modified release;
wherein the semi-solid oral dosage form contains at least 4 wt. % water, such that direct incorporation of the active ingredient into the dosage matrix without encapsulation would result in at least a 10% loss of API potency after one month at ambient storage conditions.

2. The dosage form of claim 1, wherein at least 80% of the active ingredient is encapsulated and, when analyzed by tracer diffusion, permeability testing, or confocal microscopy, the encapsulated portion shows no measurable diffusion of water or acid from the matrix into the core during a 2-week accelerated stability protocol.

3. The dosage form of claim 1, wherein the polymer encapsulating the active ingredient is simultaneously pH-sensitive, biodegradable within the gastrointestinal tract, and inert with respect to both the active ingredient and matrix excipients.

4. The dosage form of claim 1, wherein the encapsulated active ingredient is selected from APIs that are water-sensitive, acid-sensitive, oxygen-sensitive, UV-sensitive, or has an unpleasant taste.

5. The dosage form of claim 1, wherein the encapsulated active ingredient comprises a vitamin, probiotic, peptide, enzyme, RNA, mRNA, or gene therapeutic agent that is unstable in the presence of water, acid, or oxygen.

6. The dosage form of claim 1, wherein the encapsulation is selected such that the encapsulated portion is not in direct contact with water or acidifying agents present in the semi-solid matrix.

7. The dosage form of claim 1, wherein the encapsulation comprises at least two discrete polymer layers, each independently providing a barrier to a destabilizing agent selected from water, acid, oxygen, or UV light, the barrier function verified by tracer diffusion or permeability testing.

8. The dosage form of claim 1, wherein the encapsulation confers time-dependent, pH-dependent, enzyme-triggered, or smart-responsive release selected from delayed, sustained, pulsatile, or site-specific lower gastrointestinal delivery.

9. The dosage form of claim 1, wherein the encapsulating polymer comprises a blend of at least one biodegradable polyester and one time-dependent polymer.

10. The dosage form of claim 1, further comprising one or more excipients selected from flavoring agents, sweeteners, colorants, acidulants, surfactants, fillers, plasticizers, and preservatives, all within FDA-permitted limits.

11. The dosage form of claim 1, wherein the encapsulation of the active ingredient imparts reduction of unpleasant taste or odor, validated by taste-sensing analytical technology.

12. The dosage form of claim 1, wherein the encapsulated active ingredient remains stable after exposure to temperatures between 25° C. and 60° C. or relative humidity above 60% for at least one month.

13. The dosage form of claim 1, wherein the encapsulated API is selected from acetaminophen, ibuprofen, omeprazole, mesalamine, vitamin C, vitamin B12, folate, iron, calcium, probiotics, omega-3 fatty acids, or an API with any functional group prone to hydrolysis.

14. The dosage form of claim 1, wherein the encapsulation enables a tailored pharmacokinetic release profile selected from immediate, delayed, controlled, pulsatile, or site-specific release.

15. The dosage form of claim 1, wherein the excipients comprise water such that the matrix contains at least 2 wt. % water.

16. The dosage form of claim 1, wherein the encapsulating polymer comprises an antioxidant or reducing agent selected from ascorbic acid, glutathione, or tocopherol.

17. The dosage form of claim 1, wherein the encapsulation is produced by spray drying, coacervation, solvent evaporation, or fluid bed coating.

18. The dosage form of claim 1, wherein at least a portion of the API is hydrophobic, water-insoluble, or poorly bioavailable, and the encapsulation enhances its oral bioavailability.

19. The dosage form of claim 1, wherein the polymeric encapsulation is configured to degrade upon exposure to gastrointestinal enzymes or colonic bacteria for targeted colonic release.

20. The dosage form of claim 1, wherein the encapsulation and dosage form remain intact and API-stable after exposure to manufacturing temperatures up to 95° C.

21. The dosage form of claim 1, comprising two or more independently encapsulated APIs with different controlled-release profiles, in a single unit.

22. The dosage form of claim 1, wherein the dosage unit is further overcoated with an edible, water-resistant, oxygen-barrier, or taste-masking film.

23. The dosage form of claim 1, wherein the encapsulated API is co-encapsulated with an absorption enhancer.

24. The dosage form of claim 1, wherein mean content uniformity of the encapsulated portion varies by less than 10% between units.

25. The dosage form of claim 1, wherein the manufacturing process includes a step of inline or automated real-time monitoring using imaging or spectroscopy to verify encapsulation integrity, coating thickness, or content uniformity.

26. A semi-solid oral dosage form comprising an encapsulated active ingredient wherein the encapsulation fully surrounds the active ingredient, the encapsulation comprises at least one polymer selected from cellulose derivatives or methacrylic acid copolymers, and the dosage form is pharmaceutically or nutraceutically acceptable for human or animal ingestion in a format selected from gummies, oral thin films, pastilles, or chewable gels, with at least 2 wt. % water in the matrix; wherein the encapsulation forms a physical barrier such that the encapsulated active ingredient does not come in direct contact with excipients present in the semi-solid oral dosage form under manufacturing, storage, or use conditions.

27. The semi-solid oral dosage form of claim 26, wherein the encapsulation comprises at least one pH-sensitive polymer selected from the group consisting of Eudragit L100, Eudragit S100, or cellulose acetate phthalate (CAP); and is further configured to degrade or become soluble only at pH above 6.0 or in the presence of gastrointestinal enzymes or colonic bacteria, enabling targeted delivery of the active ingredient in the intestine or colon.

28. The semi-solid oral dosage form of claim 26, wherein the complete physical isolation of the encapsulated active ingredient from matrix excipients is verified by at least one analytical method selected from scanning electron microscopy, confocal microscopy, tracer diffusion assay, or permeability testing.

29. The semi-solid oral dosage form of claim 26, wherein the matrix is a gummy or oral thin film comprising at least 2 wt. % water, and the dosage form further comprises both (i) unencapsulated active ingredient for immediate release and (ii) encapsulated active ingredient for delayed, sustained, or targeted release within a single matrix.

30. The semi-solid oral dosage form of claim 26, wherein the encapsulation comprises at least two discrete polymer layers, each independently selected to provide barrier protection against a destabilizing agent selected from the group consisting of water, acid, oxygen, or UV light, as demonstrated by tracer diffusion or permeability testing.
